(19) United States
(12) Patent Application Publication
(10) Pub. No.: US 2018/0371076 A1
(43) Pub. Date: Dec. 27, 2018

(54) MATERNAL TH17 CELLS AND PSYCHIATRIC DISORDERS

(71) Applicant: New York University, New York, NY (US)

(72) Inventors: Dan R. Littman, New York, NY (US); Jun R. Huh, Newton, MA (US)

(21) Appl. No.: 15/880,911

(22) Filed: Jan. 26, 2018

Related U.S. Application Data
(60) Provisional application No. 62/450,612, filed on Jan. 26, 2017.

(51) Int. Cl. C07K 16/24; A61P 29/00; A61K 9/00

(52) U.S. Cl. CPC C07K 16/244; A61P 29/00; A61K 38/00; A61K 9/0053; A61K 9/0019

(57) ABSTRACT

The present invention provides a method for reducing the risk of developing a psychiatric disorder (e.g., ASD, of which autism is a particular example) in a fetus comprising administering an inhibitor of T helper 17 (Th17) cell activity to the mother of the fetus, while the mother is pregnant with the fetus. The invention further relates to the use and application of compounds or agents that inhibit Th17 activity or that inhibit IL-17a or IL-17 signaling for reducing fetal risk for developing a psychiatric disorder. The invention relates to compounds or agents that inhibit Th17 activity or that inhibit IL-17a for reducing fetal risk for developing a psychiatric disorder, such as ASD, and use of such compounds or agents in the preparation of a medicament for reducing fetal risk for developing a psychiatric disorder.

### CROSS-REFERENCE TO RELATED APPLICATIONS

The present application is a non-provisional application claiming the priority of copending provisional application Ser. No. 62/450,612, filed Jan. 26, 2017, the disclosure of which is incorporated by reference herein in its entirety. Applicants claim the benefits of this application under 35 U.S.C. § 119 (e).

### GOVERNMENT SUPPORT

The research leading to the present invention was funded in part by National Institutes of Health grants F31NS083277 and R00DK091508. The United States government has certain rights in the invention.

### FIELD OF THE INVENTION

The present invention relates generally to reducing the risk for developing a psychiatric disorder [e.g., autism spectrum disorder (ASD), schizophrenia, and/or depression] in a fetus. More particularly, the present invention relates to a method for reducing the risk of developing a psychiatric disorder (e.g., ASD, of which autism is a particular example) in a fetus involving administering an inhibitor of T helper 17 (Th17) cell activity to the mother of the fetus, while the mother is pregnant with the fetus at risk. Accordingly, the invention relates to the use and application of compounds or agents that inhibit Th17 activity for reducing fetal risk for developing a psychiatric disorder. In a further aspect, the invention relates to compounds or agents that inhibit Th17 activity for reducing fetal risk for developing a psychiatric disorder, such as ASD, and use of such compounds or agents in the preparation of a medicament for reducing fetal risk for developing a psychiatric disorder.

### BACKGROUND OF THE INVENTION

Accumulating evidence points to a central role for immune dysregulation in utero as a risk factor in Autism Spectrum Disorder (ASD)^{1, 2, 3}. Human studies suggest that maternal viral infections early in pregnancy correlate with an increased frequency of ASD in the offspring⁴. This observation, coined maternal immune activation (MIA), has been modeled in rodents by inducing inflammation in pregnant dams. MIA requires the pro-inflammatory effector cytokine interleukin 6 (IL-6) to produce ASD-like phenotypes in the offspring⁵. A number of other inflammatory cytokines and chemokines have, however, been implicated in MIA, including tumor necrosis factor-α (TNF-α), IL-113, and IL-8. Accordingly, information available to date demonstrates that a variety of inflammatory molecules contribute to MIA and thus, MIA is viewed as a generalized state of inflammation. See, for example, Harvey et al. (2014, Brain Behav Immun 40:27), Washington et al. (2015, Epilepsy Behav 50:40), and Ballendine et al. (2015, Prog Neuropsychopharmacol Biol Psychiatry 57:155), the entire content of each of which is incorporated herein by reference. The mechanism/s whereby inflammatory cytokines and chemokines contribute to MIA and MIA contributes to the development of autism and the specific immune cell population(s) involved are unknown.

The citation of references herein shall not be construed as an admission that such is prior art to the present invention.

### SUMMARY OF THE INVENTION

As described herein, the present inventors show that pro-inflammatory T cells expressing IL-17 cytokines are required in mothers for MIA to induce ASD-like phenotypes in affected offspring. Consistent with this observation, IL-17a blocking antibody administration in pregnant females protected against the development of MIA-induced behavioral deficits in the offspring. T cell-specific inactivation in mothers of RORγt, the transcription factor required for differentiation of T helper 17 (Th17) cells, a major source of IL-17a, similarly protected from induction of ASD-like phenotypes. Importantly, the present inventors also found abnormal cortical development in affected offspring, and this abnormality was rescued by inhibition of IL-17a signaling. These data suggest that therapeutic targeting in susceptible pregnant mothers of Th17 cells, major contributors to autoimmune disease, will reduce the likelihood of bearing children with inflammation-induced ASD phenotypes.

In accordance with the invention, a method is provided for reducing the risk of developing a psychiatric disorder in a fetus, the method comprising administering an inhibitor of T helper 17 (Th17) cell activity to a female during a pregnancy wherein the female is carrying the fetus in utero. In an aspect, the psychiatric disorder is autism spectrum disorder, schizophrenia, or depression. In a further aspect, the autism spectrum disorder is autism.

In an aspect of the invention, the psychiatric disorder is associated with maternal inflammation.

In accordance with a method of the invention, the inhibitor of Th17 cell activity is administered to the female during the first, the second or third trimester of the pregnancy. The inhibitor may be administered during the pregnancy at any such time that maternal immune activation (MIA) is identified, confirmed or suspected. Administration may be at any suitable point in pregnancy when MIA is identified, confirmed or suspected. In an aspect, an inhibitor of Th17 cell activity and/or of IL-17 activity or signaling, is administered during the first, the second or third trimester of pregnancy. In an aspect, an inhibitor of Th17 cell activity and/or of IL-17a activity or signaling, is administered during the first, the second or third trimester of pregnancy. In an aspect, an inhibitor of Th17 cell activity and/or of IL-17a activity or signaling, is administered during the first, the second and third trimester of pregnancy. In an aspect, an inhibitor of Th17 cell activity and/or of IL-17a activity or signaling, is administered in the second trimester of pregnancy. In an aspect, an inhibitor of Th17 cell activity and/or of IL-17a activity or signaling, is administered in the third trimester of pregnancy.

In an aspect of the instant method(s), the female has elevated levels of IL-17a in her sera during the pregnancy with the fetus. In an aspect of the instant method(s), the female has elevated levels of IL-17a or other IL-17 family cytokine in her sera during the pregnancy with the fetus. In an method according to the invention, a pregnant female is first evaluated for elevated levels of IL-17a in her sera during pregnancy compared to a control, including other pregnant females, and if elevated IL-17a sera levels are found, an inhibitor of Th17 cell activity and/or of IL-17a activity or signaling, is administered to said pregnant female, thereby reducing the risk of developing a psychiatric disorder, particularly autism spectrum disorder (ASD), schizophrenia, and/or depression, in the fetus or offspring of said pregnant female. In an method according to the invention, a pregnant female is first evaluated for elevated levels of IL-17a or other IL-17 family cytokine in her sera during pregnancy compared to a control, including other pregnant females, and if elevated IL-17a or other IL-17 family cytokine sera levels are found, an inhibitor of Th17 cell activity and/or of IL-17a activity or signaling and/or other IL-17 family cytokine activity or signaling, is administered to said pregnant female, thereby reducing the risk of developing a psychiatric disorder, particularly autism spectrum disorder (ASD), schizophrenia, and/or depression, in the fetus or offspring of said pregnant female. In one aspect of such method, an inhibitor of Th17 cell activity and/or of IL-17a activity or signaling and/or of other IL-17 family cytokine activity or signaling, is administered to said pregnant female, thereby reducing or blocking the manifestation of behavioral deficits, particularly behavioral deficits associated with ASD, in offspring or in a fetus. In an aspect, behavior deficits may include altered social interactions and/or repetitive behavior.

In accordance with an aspect of the method(s) of the invention, the female was or is afflicted with a hyper-inflammatory condition during the pregnancy with the fetus. In one aspect, the hyper-inflammatory condition is associated with a viral or bacterial infection or exposure to an inflammatory or environmental toxin during the pregnancy with the fetus. In an aspect, the female was or is afflicted with the hyper-inflammatory condition during the first, second, or third trimester of the pregnancy with the fetus.

The invention is directed to methods wherein the inhibitor of Th17 cell activity is administered to the female during or after the hyper-inflammatory condition. The invention is directed to methods wherein the inhibitor of IL-17a activity is administered to the female during or after the hyper-inflammatory condition. The invention is directed to methods wherein the inhibitor of IL-17a signaling is administered to the female during or after the hyper-inflammatory condition. The invention is directed to methods wherein the inhibitor of IL-17 family cytokine activity or signaling is administered to the female during or after the hyper-inflammatory condition.

The invention is directed to methods wherein the inhibitor of Th17 cell activity is administered to the female before or prior to the hyper-inflammatory condition. The invention is directed to methods wherein the inhibitor of IL-17a activity is administered to the female before or prior to the hyper-inflammatory condition, or prior to symptoms or indications of the hyper-inflammatory condition. The invention is directed to methods wherein the inhibitor of IL-17a and/or other IL-17 family cytokine activity is administered to the female before or prior to the hyper-inflammatory condition, or prior to symptoms or indications of the hyper-inflammatory condition. The invention is directed to methods wherein the inhibitor of IL-17a signaling is administered to the female before or prior to the hyper-inflammatory condition or prior to symptoms or indications of the hyper-inflammatory condition. The invention is directed to methods wherein the inhibitor of IL-17a and/or other IL-17 family cytokine signaling is administered to the female before or prior to the hyper-inflammatory condition or prior to symptoms or indications of the hyper-inflammatory condition. In an embodiment, the inhibitor of IL-17 activity is an antibody specific for IL-17a or IL-17 receptor.

In an aspect, the inhibitor of Th17 cell activity is an inhibitor of retinoic acid receptor-related orphan nuclear receptor gamma t (RORγt) activity and/or interleukin 17 (IL-17) activity or an enhancer of T regulatory (Treg) cell activity. In an embodiment, the inhibitor of RORγt activity is TMP778, SR1001, or SR2211.

In an aspect, the inhibitor of IL-17 activity is an antibody specific for a Th17 cell specific cytokine or a Th17 specific cell surface protein. In one aspect, the Th17 cell specific cytokine is IL-17f or IL-22. In an embodiment, the Th17 specific cell surface protein is CCR6.

In an aspect, the inhibitor of IL-17 activity is a human monoclonal antibody or a humanized monoclonal antibody. In one embodiment, the human monoclonal antibody is brodalumab (AMG 827). In an embodiment, the humanized monoclonal antibody is ixekizumab (LY2439821) or secukinumab (AIN457).

In one aspect of the invention, the inhibitor of IL-17 activity is an antibody specific for the p19 subunit of IL-23, the p40 subunit of IL-23 and IL-12, or the IL-23 receptor. In an embodiment, the antibody specific for the p19 subunit of IL-23 is MK-3222 (SCH 900222), CNTO 1959, or AMG 139. In an embodiment, the antibody specific for the p40 subunit of IL-23 and IL-12 is Stelara (ustekinumab; CNTO 1275).

In a method aspect of the invention, the inhibitor of Th17 cell activity is administered intravenously, subcutaneously, intraperitoneally, or orally.

In a particular aspect, the inhibitor of Th17 cell activity does not transfer across the placenta or is modified to reduce or prevent transfer across the placenta.

In one further aspect, the female does not have a pre-existing condition associated with aberrant Th17 cell activity.

In an aspect, the pre-existing condition associated with aberrant Th17 activity is multiple sclerosis, psoriasis, rheumatoid arthritis, or Crohn's Disease.

In accordance with the instant invention, a method is provided for decreasing the likelihood of a psychiatric disorder in a fetus, the method comprising administering an inhibitor of T helper 17 (Th17) cell activity to a female during a pregnancy wherein the female is carrying the fetus in utero.

In accordance with the instant invention, a method is provided for treating a pregnant female with a hyper-inflammatory condition, the method comprising administering an inhibitor of Th17 cell activity to the female while pregnant with a fetus to reduce inflammation in the pregnant female, thereby decreasing risk for developing a psychiatric disorder in the fetus.

In an aspect of the invention, a method(s) is provided for reducing ASD-like phenotypes in offspring or in a fetus, particularly wherein maternal immune activation (MIA) is identified or suspected in the pregnant female carrying the fetus. In an aspect, the method comprises administering to the pregnant female an inhibitor of T helper 17 (Th17) cell activity, an inhibitor of IL-17a activity, or an inhibitor of IL-17a signaling. In an aspect, the inhibitor is an IL-17a antibody.

In an aspect, a method is provided for reducing or protecting against the development of maternal immune activation (MIA)-induced behavioral deficits in offspring or a fetus. IN a particular aspect, the behavioral deficits associated are with ASD, in offspring or in a fetus, particularly wherein maternal immune activation (MIA) is identified or suspected in the pregnant female carrying the fetus. In an aspect, the method comprises administering to the pregnant female an inhibitor of T helper 17 (Th17) cell activity, an inhibitor of IL-17a activity, or an inhibitor of IL-17a signaling. In an aspect, the inhibitor is an IL-17a antibody.

In an aspect of the invention, a method(s) is provided for reducing behavioral deficits, particularly behavioral deficits associated with ASD, in offspring or in a fetus, particularly wherein maternal immune activation (MIA) is identified or suspected in the pregnant female carrying the fetus. In an aspect, the method comprises administering to the pregnant female an inhibitor of T helper 17 (Th17) cell activity, an inhibitor of IL-17a activity, or an inhibitor of IL-17a signaling. In an aspect, the inhibitor is an IL-17a antibody.

In an aspect of the methods of the invention, RORγt is inactivated.

In an aspect of the invention, antibody blockade of IL-17a activity in mothers protects against the development of MIA-induced behavioral abnormalities in offspring.

In an aspect of the instant methods, atypical cortical development in offspring is rescued, reduced or inhibited by inhibition of T helper 17 (Th17) cell activity, inhibition of IL-17a activity, or inhibition of IL-17a signaling.

Other objects and advantages will become apparent to those skilled in the art from a review of the following description which proceeds with reference to the following illustrative drawings.

### BRIEF DESCRIPTION OF THE DRAWINGS

FIG. 1A-1D. IL-17a is elevated in the serum and placenta/decidua of pregnant mothers. A, Serum concentrations of IL-6 (n=3˜5 mice per group, 2 independent experiments) at 3 or 24 h after PBS or poly(I:C) injection into pregnant dams. B, Serum and placenta/decidua concentrations of IL-17a (CD3: n=7, others: n=17˜28 mice per group) at E14.5/E15.5. CD3 refers to anti-CD3 antibody injection, which was used as a positive control. Nonpreg refers to non-pregnant females and pla to decidua/placenta lysate. C, D, Supernatant concentrations of IL-17a from ex vivo cultured mononuclear cells, isolated from placenta/decidua (C) or duodenum (D) of PBS- or poly(I:C)-treated mice at E14.5/E15.5. Each sample represents an individual animal. A, B, One-way ANOVA with Holm-Sidak corrections (**p<0.01 and *p<0.05). C, D, Non-parametric Mann-Whitney U-test (*p<0.05). Graphs show mean+/−s.e.m.

FIG. 2A-2D. The IL-17a pathway promotes ASD-like phenotypes in the offspring of pregnant dams following MIA. A, Schematic diagram of experimental design: pregnant mothers of the indicated genetic background (or subjects of antibody treatment) were injected with PBS or poly(I:C) to induce MIA. B, Ultrasonic vocalization (USV) assay. At P9, pups from the indicated conditions were separated from their mothers to elicit USV calls. The numbers of pup calls are plotted on the y-axis (n=10˜11 pups for PBS-treated groups, n=23˜25 for Poly(I:C)-treated groups) ; n=5˜15 mice per cohort combined from 2˜5 independent experiments. C, Social approach behavior, graphed as social preference index (percentage time spent in close proximity to a stimulus mouse out of total investigation time); n=6 and 4 adult offspring for PBS-treated mothers administered isotype control or anti-IL-17a antibody, respectively; and n=7 and 9 for poly(I:C)-treated mothers that received isotype control or anti-IL-17a antibody. D, Marble burying behavior. Percentage of the number of buried marbles is plotted on the y-axis (n=6 and 4 for adult offspring for PBS-treated mothers administered isotype control or anti-IL-17a antibody, respectively; and n=7, and 10 offspring of poly(I:C)-treated dams that received isotype control or anti-IL-17a antibody, respectively). **p<0.01. One-way ANOVA with Holm-Sidak corrections. Graphs show mean+/−s.e.m.

FIG. 3A-3C. RORγt expression in T cells of pregnant dams subjected to MIA is required for ASD-like phenotypes in the offspring. A, Ultrasonic vocalization assay. USV calls of pups from PBS-versus poly(I:C)-treated mothers at P9 (n=15 (WT), 18 (RORγt HET), and 15 (RORγt KO) pups from PBS-treated mothers and n=15 (WT), 11 (RORγt HET), and 28 (RORγt KO) from poly(I:C)-treated mothers). RORγt HET and RORγt KO refer to RORγ^{Neo/+}; CD4-Cre/+ and RORγt^{floxed}/RORγ^{Neo}; CD4-Cre/+, respectively. B, Social approach behavior is graphed as social preference index or percentage of time spent investigating social stimulus. 50% social preference index indicates chance level of investigation (n=14 (WT), 15 (RORγt HET), and 3 (RORγt KO) adult offspring from PBS-treated mothers and n=27 (WT), 7 (RORγt HET), and 21 (RORγt KO) from poly(I:C)-treated mothers. c, Marble burying behavior is graphed as the percentage of buried marbles (n=11 and 19 adult offspring from PBS-treated B6 and RORγt HET mothers; n=23, 10 and 25 from poly(I:C)-treated WT, RORγt HET and RORγt KO mice per group). n=5˜15 mice per cohort combined from 4˜7 independent experiments. **p<0.01 and *p<0.05. One-way ANOVA with Holm-Sidak corrections. Graphs show mean+/−s.e.m.

FIG. 4A-4B. Abnormal cortical development in embryos from mothers with MIA. A, SATB2 immunofluorescent staining of E14.5 fetal brain, derived from PBS- or poly(I:C)-injected mothers, pretreated with isotype control or IL-17a blocking antibodies. Images are representative of two independent experiments. A, SATB2 (a marker more intensely expressed in the upper part of the cortex) and TBR1(a marker restricted to the lower part of the cortex) staining of E18.5 fetal brain from animals treated as in A. Images are representative of two independent experiments. Images from additional fetuses are shown in Extended Data FIGS. 6 and 7. (MZ: marginal zone, CP: cortical plate, SP: subplate, SVZ: subventricular zone, VZ: ventricular zone, IZ: intermediate zone).

FIG. 5A-5E. Blocking IL-17a pathway at E14.5 rescues abnormal communication and repetitive behavioral phenotypes in the offspring. A, Schematic diagram of experimental design: pregnant mothers were injected with PBS or poly(I:C) at E12.5 to induce MIA, followed by isotype or anti-IL-17a Ab injection at E14.5. B, Ultrasonic vocalization (USV) assay. At P9, pups from the indicated conditions were separated from their mothers to elicit USV calls. C, Social approach behavior is graphed as a social preference index or percentage of time spent investigating social versus inanimate stimuli. 50% social preference index indicates chance level of investigation. D, Marble burying behavior. Percentage of the number of buried marbles is plotted on the y-axis. E, Total distance moved by offspring in the social approach test. B-E, Graphs show mean+/−s.e.m. Each symbol represents individual animal. *p<0.01 and **p<0.05.

FIG. 6A-6D. Th17 cells in the placenta and decidua of pregnant mice experiencing MIA. A, B, C, Flow cytometry of CD4⁺ T cells stained intracellularly for IL-17a and IFN-γ. Mononuclear cells were collected from placenta/decidua of PBS- or poly(I:C)-treated pregnant mice at E14.5 and E15.5. The cells were stimulated for 4-5 h with PMA/Ionomycin and stained for surface markers and intracellular cytokines. Each symbol represents an individual mouse. RORγ/γt KO refers to a germline deletion mutant removing both RORγ and RORγt (RORγ^{Neo/Neo}). Statistical analysis was by a two-tailed unpaired Student's t test, *p<0.05. D, Serum and placenta/decidua concentrations of IL-10 at E15.5 (n=5˜10 mice per group). One-way ANOVA with Holm-Sidak corrections (**p<0.01). Graphs show mean+/−s.e.m.

FIG. 7. Offspring of anti-IL-17a treated mice exhibit normal locomotor activity. Social approach behavior, graphed as their total distance moved; n=6 and 4 adult offspring for PBS-treated mothers administered isotype control or anti-IL-17a antibody, respectively; and n=7 and 9 for poly(I:C)-treated mothers that received isotype control or anti-IL-17a antibody.

FIG. 8A-8D. Critical role of maternal RORγ/γt for exploratory behavioral deficit in MIA-affected offspring. A, B, Open field test. Offspring of WT and RORγ/γt HET mice treated with poly(I:C) spent less time in the center compared with offspring of PBS-treated WT or poly(I:C)-treated RORγ/γt KO mothers (A), but traveled similar total distances (B) as control offspring of PBS-treated mothers in the open field arena (n=17 and 13 adult offspring from PBS- and poly(I:C)-treated B6 mice per group: n=5 and 10 from poly(I:C)-treated RORγ/γt HET and RORγ/γt KO mice per group). RORγ/γt KO refers to a germline deletion mutant removing both RORγ and RORγt (RORc^{Neo/Neo}). One-way ANOVA with Holm-Sidak corrections (*p<0.05). Graphs show mean+/−s.e.m. C, D, Representative tracks, recorded during the open-field session, for adult offspring of poly(I:C)-treated RORγ/γt Het and RORγ/γt KO mice.

FIG. 9A-9B. Offspring of maternal RORγt KO mice exhibit normal locomotor activity. Offspring tested for marble burying behavior show comparable total distance moved (A) and velocity (B), regardless of their maternal genotypes and treatments (n=11 and 19 adult offspring from PBS-treated WT and RORγt HET mothers; n=23, 10 and 25 from poly(I:C)-treated WT, RORγt HET and RORγt KO mice per group). n=5˜15 mice per cohort combined from 4-7 independent experiments. RORγt HET and RORγt KO refer to ROR^{Neo/+}; CD4-Cre/+ and RORγt^{floxed}/RORγ^{Neo}: CD4-Cre/+, respectively.

FIG. 10A-10B. Enhanced IL-17Ra expression in the fetal brain following MIA induction in the mother. In situ hybridization with an Il17ra RNA probe in E14.5 fetal brains derived from PBS-(A) or poly(I:C)-(B) injected mothers. Images are representative of two independent experiments. Arrowheads denote enhanced IL-17a expression in the cortical area of the fetal brain. * indicates artifacts.

FIG. 11. SATB2 expression in the fetal brain following MIA induction in the mother with or without IL-17a blocking antibody treatment. SATB2 immunofluorescence staining of E14.5 fetal brain, derived from PBS- or poly(I:C)-injected mothers, pretreated with isotype control or IL-17a blocking antibodies. Images in each row are from individual fetuses that are from two independent experiments.

FIG. 12. SATB2 and TBR1 expression in the fetal brain following MIA induction in the mother with or without IL-17a blocking antibody treatment. SATB2 and TBR1 immunofluorescence staining of E18.5 fetal brain, derived from PBS- or poly(I:C)-injected mothers, pretreated with isotype control or IL-17a blocking antibodies. Images in each row are from individual fetuses that are from two independent experiments.

FIG. 13. Abnormal cortical development in adult offspring of mothers subjected to MIA. Coronal sections of adult offspring brain (P60), derived from PBS- or poly(I:C)-treated mothers, pretreated with isotype control or IL-17a blocking antibodies. Immunofluorescence staining with TBR1 (a marker for layers 2, 3, 5 and 6), SATB2 (a marker more intensely expressed in upper layers), and CTIP2 (a marker restricted to layers 4-6) antibodies. Arrowheads indicate abnormal staining patterns in the cortex.

FIG. 14A-14I. IL-17a increase in mothers subjected to MIA leads to elevated IL-17Ra mRNA expression in the offspring. (A) Serum concentrations of IL-6 (n=3-5 mice per group, 2 independent experiments) at 3 or 24 h after PBS or poly(I:C) injection into pregnant dams at E12.5. (B) Serum concentrations of maternal IL-17a (n=4-8 mice per group, 2 independent experiments) at E14.5 in PBS- or poly(I:C)-injected mothers, pretreated with or without IL-17a blocking antibodies. (C and D) Relative IL-6 (C) and IL-17a (D) mRNA expression in cells isolated from placenta/decidua of PBS- or poly(I:C)-treated mothers at E14.5 and cultured in vitro for 24 h. The results are representative of three independent experiments. For each probe set, relative mRNA expression of one biological replicate from PBS-treated dams was set at 1. Real-time PCR analysis of relative expression of indicated genes compared to the level of Gapdh in cells from PBS-treated dams. (E) Supernatant concentrations of IL-17a from ex vivo cultured mononuclear cells, isolated from placenta/decidua of PBS- or poly(IC)-treated pregnant dams. Stim refers to PMA and Ionomycin stimulation. (F and G) Relative IL-17Ra (F) and IL-17Rc (G) mRNA levels in E14.5 male fetal brain, derived from PBS- or Poly(I:C)-injected mothers, pretreated with isotype control (Cont) or IL-17a blocking antibodies (anti-IL-17a). The relative mRNA fold change, compared to the PBS and Cont-treated group, is plotted on the y-axis (n=7 (PBS, Cont), n=7 (PBS, anti-IL-17a), n=7 (Poly(I:C), Cont), n=7 (Poly(I:C), anti-IL-17a); from 2-3 independent experiments). (H) In situ hybridization with an IL-17Ra RNA probe in E14.5 male fetal brains derived from PBS- or poly(I:C)-injected mothers. Images are representative of four independent experiments. (I) Relative signal intensity for images shown in (H). Scale bar represents 100 μm. (A, B, E, F and G) One-way ANOVA with Tukey post-hoc tests. (C, D and I) Student's t test. **p<0.01. Graphs show mean+/−s.e.m.

FIG. 15A-15H. The IL-17a pathway promotes abnormal cortical development in the offspring of pregnant dams following MIA. (A) Immuno-fluorescence staining of SATB2 (a marker of postmitotic neurons in superficial cortical layers) in E14.5 male fetal brain, derived from PBS- or poly(I:C)-injected mothers, pretreated with isotype control (Cont) or IL-17a blocking antibodies (anti-IL-17a). (MZ: marginal zone, CP: cortical plate, SP: subplate, SVZ: subventricular zone, VZ: ventricular zone). (B) Staining of SATB2 and TBR1 (a marker restricted to deeper cortical layers) in E18.5 male fetal brains from animals treated as in (A). II-IV, V and VI refer to different cortical layers. (C) Quantification of SATB2 intensity in the cortical plate of E14.5 fetal brains (n=8 (PBS, Cont), n=8 (PBS, anti-IL-17a), n=8 (Poly(I:C), Cont), n=8 (Poly(I:C), anti-IL-17a), 3 independent experiments). (D) Quantification of TBR1 and SATB2 positive cells in a 300×300 μm² region of interest (ROI) centered on the malformation in the cortical plate of E18.5 fetal brains (n=20 (PBS, Cont), n=20 (PBS, anti-IL-17a), n=24 (Poly(I:C), Cont), n=20 (Poly(I:C), anti-IL-17a), 5 independent experiments). (E) The spatial location of the cortical patch in E18.5 male fetal brains from poly(I:C)-injected mothers pretreated with control antibodies (n=20 (Poly(I:C), Cont)). (F) The disorganized patches of cortex observed in fetuses from poly(I:C)-injected mothers were categorized into groups based on morphology: Protrusions, intrusions or other abnormal patterns and their representative images are shown. (G) Percentage of the cortical patches in each category (n=24 (Poly(I:C), Cont)). (H) Thickness of the cortical plate in E18.5 fetal brains, derived from PBS- or poly(I:C)-injected mothers, pretreated with isotype control or IL-17a blocking antibodies (n=20 (PBS, Cont), n=20 (PBS, anti-IL-17a), n=20 (Poly(I:C), Cont), n=20 (Poly(I:C), anti-IL-17a), 5 independent experiments). (A, B and F) Scale bar represents 100 μm. One-way ANOVA (C and H) and Two-way ANOVA (D) with Tukey post-hoc tests. **p<0.01 and *p<0.05. Graphs show mean+/−s.e.m.

FIG. 16A-16D. The IL-17a pathway promotes ASD-like phenotypes in the MIA offspring. (A) Ultrasonic vocalization (USV) assay. At P9, pups from the indicated experimental groups were separated from their mothers to elicit USV calls. The number of pup calls is plotted on the y-axis (n=25 (PBS, Cont), n=28 (PBS, anti-IL-17a), n=38 (Poly(I:C), Cont), n=34 (Poly(I:C), anti-IL-17a); from 6-7 independent experiments. (B) Social approach behavior. Graphed as a social preference index (% time spent investigating social or inanimate stimulus out of total object investigation time) (n=15 (PBS, Cont), n=15 (PBS, anti-IL-17a), n=16 (Poly(I:C), Cont), n=20 (Poly(I:C), anti-IL-17a); from 6-7 independent experiments. (C) Marble burying behavior. Percentage of the number of buried marbles is plotted on the y-axis (n=15 (PBS, Cont), n=15 (PBS, anti-IL-17a), n=15 (Poly(I:C), Cont), n=20 (Poly(I:C), anti-IL-17a); from 6-7 independent experiments. (D) Total distance traveled during social approach behavior. (A, C and D) One-way ANOVA with Tukey post-hoc tests. (B) Two-way ANOVA with Tukey post-hoc tests. **p<0.01 and *p<0.05. Graphs show mean+/−s.e.m.

FIG. 17A-17F. RORγt expression in maternal T cells is required for manifestation of ASD-like phenotypes in the MIA model. (A) SATB2 and TBR1 staining in the cortex of E18.5 fetal brains following MIA induction with poly(I:C) in mothers with the indicated genotypes. II-IV, V and VI refer to different cortical layers. Images are representative of three independent experiments. Scale bar represents 100 μm. (B) Quantification of TBR1 and SATB2 positive cells in a 300×300 μm² ROI centered on the malformation in the cortical plate of E18.5 male fetal brains (n=6 (PBS, WT), n=6 (Poly(I:C), WT), n=6 (Poly(I:C), RORγt-TKO)). (C) Number of ultrasonic vocalizations (USV)s emitted by P9 pups. Total USVs emitted during test period (3 min) are plotted on the y-axis (n=16, 18 and 15 offspring from PBS-treated WT, RORγt HET and RORγt TKO mothers; n=15, 11 and 28 from poly(I:C)-treated WT, RORγt HET and RORγt TKO mothers); data from 4-7 independent dams. (D) Social approach behavior is graphed as a social preference index (% time spent investigating social or inanimate stimulus/total exploration time for both objects). (n=21, 15 and 15 adult offspring from PBS-treated WT, RORγt HET and RORγt TKO mothers; n=36, 15 and 21 from poly(I:C)-treated WT, RORγt HET and RORγt TKO mothers); data from 4-7 independent dams. (E) Marble burying behavior is graphed as the percentage of buried marbles. (n=14, 19 and 15 adult offspring from PBS-treated WT, RORγt HET and RORγt TKO mothers; n=32, 15 and 25 from poly(I:C)-treated WT, RORγt HET and RORγt TKO mice per group); data from 4-7 independent dams. (F) Total distance moved by offspring tested for social behavior and marble burying. RORγt HET and RORγt TKO refer to RORγ^{Neo/+}; CD4-Cre/+ and RORγt^{FL}/RORγ^{Neo}; CD4-Cre/+, respectively. (C) One-way ANOVA with Holm-Sidak post-hoc tests. (B and D) Two-way ANOVA with Tukey post-hoc tests. (E and F) One-way ANOVA with Tukey post-hoc tests. ***p<0.001, **p<0.01 and *p<0.05. Graphs show mean+/−s.e.m.

FIG. 18A-18I. IL-17a administration to the fetus promotes abnormal cortical development and ASD-like behavioral phenotypes. (A) Schematic diagram of the experimental method. Each embryo was injected intraventricularly at E14.5 with PBS or recombinant IL-17a protein mixed with Fastgreen dye. (B) SATB2 and TBR1 staining in the cortex of E18.5 male fetal brains treated as in (A). Images are representative of five independent experiments. (C) Quantification of TBR1 and SATB2 positive cells in a 300-μm wide ROI corresponding to the region of the cortical plate containing the malformation in E18.5 male fetal brain (n=20 (PBS), n=20 (IL-17a)). (D) The spatial location of the disorganized cortical patch in E18.5 fetal brain (n=20 (IL-17a)). (E) Percentage of the cortical patches in each category (n=20 (IL-17a)). (F) Ultrasonic vocalization (USV) assay. The number of pup calls is plotted on the y-axis (n=15 (PBS), n=17 (IL-17a); from 5-6 independent dams per treatment). (G) Social approach behavior. Graphed as a social preference index (% time spent investigating social or inanimate stimulus out of total object investigation time) (n=12 (PBS), n=18 (IL-17a), from 5-6 independent experiments). (H) Marble burying behavior. Percentage of the number of buried marbles is plotted on the y-axis (n=12 (PBS), n=18 (IL-17a), from 5-6 independent experiments). (I) Total distance traveled during social approach test. (C) Two-way ANOVA with Tukey post-hoc tests. (F, H and I) Student's t tests. (G) One-way ANOVA with Tukey post-hoc test. **p<0.01, *p<0.05, and ns; not significant. Graphs show mean+/−s.e.m.

FIG. 19A-19E. Therapeutic effects of blocking IL-17a signaling in pregnant dams. (A) Schematic diagram of the experimental design. At E12.5, pregnant mothers were injected with PBS or poly(I:C) to induce MIA. Two days later (E14.5), the pregnant mothers were treated with isotype or anti-IL-17a blocking antibodies. At P7˜P9, pups were separated from the mothers to measure USV calls. At ˜8wks, male offspring were subjected to the social approach test and marble burying test. (B) Ultrasonic vocalization (USV) assay. The number of pup calls is plotted on the y-axis (n=17 (PBS+Cont), n=17 (Poly(I:C)+Cont), n=27 (Poly(I:C)+anti-IL-17a; from 3-4 independent dams per treatment). (C) Social approach behavior. Graphed as a social preference index (% time spent investigating social or inanimate stimulus out of total object investigation time) (n=12 (PBS+Cont), n=10 (Poly(I:C)+Cont), n=17 (Poly(I:C)+anti-IL-17a; from 3-4 independent dams per treatment). (D) Marble burying behavior. Percentage of the number of buried marbles is plotted on the y-axis (n=12 (PBS+Cont), n=10 (Poly(I:C)+Cont), n=17 (Poly(I:C)+anti-IL-17a; from 3-4 independent dams per treatment). (E) Total distance traveled during social approach behavior. (B, D and E) One-way ANOVA with Tukey post-hoc tests. (C) Two-way ANOVA with Tukey post-hoc test. **p<0.01 and *p<0.05. Graphs show mean+/−s.e.m.

FIG. 20A-20F. Expression of multiple cytokines detected upon MIA. (A, B, C) Maternal serum concentrations of TNF-α, IFN-β and IL-1β (n=3-6 mice per group, pooled from two independent experiments) at 3, 24, 48 or 96 h after PBS or poly(I:C) injection of pregnant dams. (D) Serum and placenta/decidua concentrations of IL-10 at E15.5 (n=5˜10 mice per group). (E) Serum concentrations of maternal IL-17a (n=5-8 mice per group, pooled from two independent experiments) at E14.5 in WT or IL-6 KO mothers injected with PBS, recombinant IL-6 (mIL-6), or poly(I:C). (F) Supernatant concentrations of IL-17a from ex vivo cultured mononuclear cells, isolated from duodenum of PBS- or poly(I:C)-treated pregnant dams. Stim refers to PMA and Ionomycin stimulation. One-way ANOVA with Tukey post-hoc tests. ***P<0.001, **P<0.01, *P<0.05 and ns; not significant Graphs show mean+/−s.e.m.

FIG. 21. In situ hybridization for Il17ra in fetal brains. In situ hybridization with an il17ra RNA probe in E14.5 WT mice (left) and IL-17Ra KO (middle) fetal brains derived from poly(I:C)-injected dams. Amplification control using gapdh RNA probe in E14.5 IL-17Ra KO fetal brain derived from poly(I:C)-injected dam (right). Scale bar represents 100 μm.

FIG. 22A-22B. Schematic diagram of the experimental design. (A) Pregnant mothers of the indicated genetic background (or subjects of antibody treatment) at E12.5 were injected with PBS or poly(I:C) to induce MIA. (B) At E12.5, pregnant mothers were pretreated with isotype or anti-IL-17a blocking antibodies. 8 hours after the pretreatment, the mothers were injected with PBS or poly(I:C) to induce MIA. For histological analyses of cortical phenotypes, fetuses were sacrificed at E14.5 and E18.5. At P7 or P9, pups were separated from the mothers to measure USV calls. At ˜8wks, male offspring were subjected to the social approach test, which included 15 min of habituation over two consecutive days. At ˜9wks, the male offspring were subjected to the marble burying test.

FIG. 23A-23B. Abnormal cortical development in adult offspring of mothers subjected to MIA. (A) Coronal sections of the brains from adult offspring (P60) derived from PBS- or poly(I:C)-injected mothers, pretreated with isotype control or IL-17a blocking antibodies. Immunofluorescence staining with TBR1 (a marker for cortical layers 2, 3, 5 and 6), SATB2 (a marker more intensely expressed in superficial cortical layers), and CTIP2 (a marker restricted to layers 4-6) antibodies. Arrowheads indicate abnormal staining patterns in the cortex. Scale bar represents 100 μm. (B) Quantification of TBR1-, SATB2-, and CTIP2-positive cells in a 300-μm wide ROI centered on the malformation in the cortex of the adult brain (TBR1+ cells: n=4 (PBS, Cont), n=4 (PBS, anti-IL-17a), n=4 (Poly(I:C), Cont), n=4 (Poly(I:C), anti-IL-17a); SATB2+ cells: n=10 (PBS, Cont), n=10 (PBS, anti-IL-17a), n=10 (Poly(I:C), Cont), n=10 (Poly(I:C), anti-IL-17a); CTIP2+ cells: n=4 (PBS, Cont), n=4 (PBS, anti-IL-17a), n=4 (Poly(I:C), Cont), n=4 (Poly(I:C), anti-IL-17a)). Two-way ANOVA with Tukey post-hoc tests. **P<0.01. Graphs show mean+/−s.e.m.

FIG. 24A-24C. Properties of the litter are not affected by poly(I:C) or anti-IL-17a treatment. (A and B) Both gender ratio (A) and size (B) of litters following PBS versus poly(I:C) and control versus anti-IL17a treatments were measured upon weaning (n=68 (PBS, Cont), n=49 (PBS, anti-IL-17a), n=56 (Poly(I:C), Cont), n=61 (Poly(I:C), anti-IL-17a); from 8-9 dams per treatment). (C) Weight of the offspring from pregnant dams treated as in (A) and (B). 13˜15-week-old male mice were used for measuring weights (n=20 (PBS, Cont), n=17 (PBS, anti-IL-17a), n=18 (Poly(I:C), Cont), n=17 (Poly(I:C), anti-IL-17a)). (A, B and C) One-way ANOVA with Tukey post-hoc tests. Graphs show mean+/−s.e.m.

FIG. 25A-25F. Th17 cells in the placenta and decidua of pregnant mice experiencing MIA. (A-C) Flow cytometry of CD4+ T cells stained intracellularly for IL-17a and IFN-γ. Mononuclear cells were collected from placenta/decidua of PBS- or poly(I:C)-treated pregnant mice at E15.5 and E16.5. RORγ KO refers to a germline deletion mutant removing both RORγ and RORγt (RORγ^{Neo/Neo}). (D, E) Flow cytometry of CD4+ T cells stained intracellularly for IL-17a (Th17) and FoxP3 (Treg). Mononuclear cells were collected from placenta/decidua of PBS- or poly(I:C)-treated pregnant mice at E14.5. (AD) The cells were stimulated for 4-5 h with PMA/Ionomycin and stained for surface markers and intracellular cytokines. Each symbol represents an individual mouse. (A-E) Th17 refers to CD4+TCR-β+IL-17a+IFN-γ+/−, Th1 to CD4+TCR-β+IL-17a-IFN-γ+ and Treg to CD4+TCR-β+FoxP3+ cells. (F) Serum concentrations of maternal IL-17a (n=4 mice per group) at E18.5 in PBS- or poly(I:C)-injected WT or RORγt TKO mothers. (A and D) Student's t test. (F) One-way ANOVA with Tukey post-hoc tests.***P<0.001 and *P<0.05. Graphs show mean+/−s.e.m.

FIG. 26A-26C. Generation of RORγ/γt conditional knockout mice. (A) Schematic diagram of targeting strategy and affected allele. (B) Southern blot analysis with ES cell genomic DNA after homologous recombination. Southern blot with Probe A following EcoRV (RV) digestion produced an 8.9 kb band for WT and a 7.3 kb band for the targeted allele. Southern blot with Probe B following EcoRI (RI) digestion produced a 15.1 kb band for WT and a 6.5 kb band for the targeted allele. (C) Southern blot with Probe A following EcoRV and XhoI (XH) digestion confirmed Cre-dependent generation of the mutant allele (Conditional allele: 7.4 kb; mutant allele: 5.6 kb). DNA was prepared from the RORγt conditional ES cells, with or without Cre transfection.

FIG. 27A-27B. Genetic removal of maternal IL-17Ra rescues deficits in social approach behavior in the offspring of pregnant dams following MIA. (A) Social approach behavior is plotted as a social preference index: time spent in close proximity to a stimulus mouse as percentage of total time directed toward both the social and inanimate objects. (n=10 (PBS, IL-17Ra-HET), n=7 (Poly(I:C), WT), n=24 (Poly(I:C), IL-17Ra-HET), n=28 (Poly(I:C), IL-17Ra-KO)). (B) Serum concentrations of maternal IL-17a (n=5˜8 mice per group, pooled from two independent experiments) at E14.5 in PBS- or poly(I:C)-injected WT or IL-17Ra HET mothers. (A) Two-way ANOVA with Tukey post-hoc testing. (B) One-way ANOVA with Tukey post-hoc testing. ***P<0.001 and **P<0.01. Graphs show mean+/−s.e.m.

FIG. 28A-28B. Characterization of the disorganized cortical patch from intra-ventricular administration of IL-17a. (A) SATB2 and TBR1 staining of E18.5 fetal brains from animals treated as in (FIG. 4A). Images are representative of five independent experiments. (B) Thickness of the cortical plate in E18.5 fetal brains. (A and B) (i), (ii) and (iii) indicate subdivisions resulting from equally dividing the cortex perpendicularly through the cortical plate. Scale bar represents 100 μm. (B) Student's t test. **P<0.01, *P<0.05, and ns; not significant. Graphs show mean+/−s.e.m.

FIG. 29A-29G. IL-17a acts downstream of IL-6 in the MIA model. (A) SATB2 and TBR1 staining of the cortex in E18.5 fetal brains. PBS, IL-6 or IL-17a were intraventricularly injected into the fetal brain of the indicated genotypes at E14.5. Images are representative of 2-3 independent experiments. (B) Quantification of TBR1 and SATB2 positive cells in a 300×300 μm² ROI centered on the malformation in the cortical plate (n=6 (PBS, WT dam), n=6 (IL-6, WT dam), n=6 (IL-17a, IL-17Ra KO dam); from 2-3 independent dams per treatment). (C) Ultrasonic vocalization (USV) assay. At P9, pups from the indicated conditions in (A) were separated from their mothers to elicit USV calls. The number of pup calls is plotted on the y-axis (n=10 (PBS, WT dam), n=14 (IL-6, WT dam), n=20 (IL-17a, IL-17Ra KO dam); from 2-3 independent dams per treatment). (D) SATB2 and TBR1 staining in the cortex of E18.5 fetal brain, derived from PBS- or IL-6-injected mothers, pretreated with isotype control (Cont) or IL-17a blocking antibodies (anti-IL-17a). (E) Quantification of TBR1 and SATB2 positive cells in a 300×300 μm² ROI centered on the cortical plate containing the cortical patch (n=6 (PBS, Cont), n=6 (IL-6, Cont), n=6 (IL-6, anti-IL-17a); from 2-3 independent dams per treatment). (F) USV assay for the pups from the indicated conditions as (D) (n=10 (PBS, Cont), n=19 (IL-6, Cont), n=18 (IL-6, anti-IL-17a); from 3-4 independent dams per treatment). (G) USV assay for the pups injected with PBS or IL-17a and derived from IL-6 KO mothers injected with Poly(I:C) (n=8 (PBS, Poly(I:C)), n=10 (IL-17a, Poly(I:C)); from 2 independent dams per treatment). (B and E) Two-way ANOVA with Tukey post-hoc tests. (C and F) One-way ANOVA with Tukey post-hoc tests. (G) Student's t test. **P<0.01, *P<0.05 and ns; not significant. Graphs show mean+/−s.e.m.

FIG. 30. A proposed mechanism by which maternal Th17 cells and IL-17a induce MIA-dependent behavioral and cortical abnormalities in offspring.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

In a broad aspect, methods are disclosed herein for reducing a fetus' risk of developing a psychiatric disorder [e.g., autism spectrum disorder (ASD), schizophrenia, and/or depression], wherein such methods call for administering inhibitors of Th17 cell activity to the female carrying the fetus in utero (i.e., the fetus' mother) during the pregnancy. Such methods encompass reducing the risk of developing autism in a fetus in mammals and, more particularly, in humans, using agents or compounds that inhibit Th17 activity. In a particular aspect, methods are provided for reducing the risk of developing a psychiatric disorder in a fetus, comprising administration of one or more agents or compounds that inhibit Th17 cell activity to the fetus' mother while the mother is pregnant with the fetus. The use of one or more agents or compounds that inhibit Th17 cell activity for reducing the risk of developing a psychiatric disorder in a fetus is also encompassed herein as is its/their use in the preparation of a medicament for reducing risk of developing a psychiatric disorder in a fetus. Such agents and compounds inhibit Th17 cell activity in the pregnant mother carrying the at risk fetus and in so doing, reduce the risk of a psychiatric disorder in the fetus.

In accordance with the instant invention, a method is provided for decreasing the likelihood of a psychiatric disorder in a fetus, the method comprising administering an inhibitor of T helper 17 (Th17) cell activity to a female during a pregnancy wherein the female is carrying the fetus in utero. In accordance with the instant invention, a method is provided for treating a pregnant female with a hyper-inflammatory condition, the method comprising administering an inhibitor of Th17 cell activity to the female while pregnant with a fetus to reduce inflammation in the pregnant female, thereby decreasing risk for developing a psychiatric disorder in the fetus. In an aspect of the invention, a method(s) is provided for reducing ASD-like phenotypes in offspring or in a fetus, particularly wherein maternal immune activation (MIA) is identified or suspected in the pregnant female carrying the fetus. In an aspect, the method comprises administering to the pregnant female an inhibitor of T helper 17 (Th17) cell activity, an inhibitor of IL-17a activity, or an inhibitor of IL-17a signaling. In an aspect, the inhibitor is an IL-17a antibody.

In an aspect, a method is provided for reducing or protecting against the development of maternal immune activation (MIA)-induced behavioral deficits in offspring or a fetus. In a particular aspect, the behavioral deficits associated are with ASD, in offspring or in a fetus, particularly wherein maternal immune activation (MIA) is identified or suspected in the pregnant female carrying the fetus. In an aspect, the method comprises administering to the pregnant female an inhibitor of T helper 17 (Th17) cell activity, an inhibitor of IL-17a activity, or an inhibitor of IL-17a signaling. In an aspect, the inhibitor is an IL-17a antibody.

In an aspect of the invention, a method(s) is provided for reducing behavioral deficits, particularly behavioral deficits associated with ASD, in offspring or in a fetus, particularly wherein maternal immune activation (MIA) is identified or suspected in the pregnant female carrying the fetus. In an aspect, the method comprises administering to the pregnant female an inhibitor of T helper 17 (Th17) cell activity, an inhibitor of IL-17a activity, or an inhibitor of IL-17a signaling. In an aspect, the inhibitor is an IL-17a antibody. In an aspect of the methods of the invention, RORγt is inactivated. In an aspect of the invention, antibody blockade of IL-17a activity in mothers protects against the development of MIA-induced behavioral abnormalities in offspring.

Autism spectrum disorder (ASD) refers to a condition related to brain development that impacts how a person/animal/human perceives and socializes with others, causing problems in social interaction and communication. The disorder also includes limited and repetitive patterns of behavior. The term “spectrum” in autism spectrum disorder refers to the wide range of symptoms and severity. Autism spectrum disorder includes conditions that were previously considered separate—autism, Asperger's syndrome, childhood disintegrative disorder and an unspecified form of pervasive developmental disorder. Asperger's syndrome is generally thought to be at the mild end of autism spectrum disorder.

People/animals/humans with ASD often have these characteristics: ongoing social problems that include difficulty communicating and interacting with others; repetitive behaviors as well as limited interests or activities; symptoms that typically are recognized in the first two years of life; symptoms that hurt the individual's ability to function socially, at school or work, or other areas of life. There are two main types of behaviors: restricted/repetitive behaviors and social communication/interaction behaviors. Restrictive/repetitive behaviors may include: repeating certain behaviors or having unusual behaviors; having overly focused interests, such as with moving objects or parts of objects; and/or having a lasting, intense interest in certain topics, such as numbers, details, or facts. Social communication/interaction behaviors may include: getting upset by a slight change in a routine or being placed in a new or overly stimulating setting; making little or inconsistent eye contact; having a tendency to look at and listen to other people less often; rarely sharing enjoyment of objects or activities by pointing or showing things to others; responding in an unusual way when others show anger, distress, or affection; failing to, or being slow to, respond to someone calling their name or other verbal attempts to gain attention; having difficulties with the back and forth of conversations; often talking at length about a favorite subject without noticing that others are not interested or without giving others a chance to respond; repeating words or phrases that they hear; using words that seem odd, out of place, or have a special meaning known only to those familiar with that person's way of communicating; having facial expressions, movements, and gestures that do not match what is being said; having an unusual tone of voice that may sound sing-song or flat and robot-like; having trouble understanding another person's point of view or being unable to predict or understand other people's actions.

In accordance with the invention, an MIA model was utilized and ASD-like phenotypes were evaluated in the animal model. ASD-like phenotypes that serve to manifest disease aspects or indicators for evaluation of ASD include restricted/repetitive behaviors and social communication/interaction behaviors. The ASD-like phenotypes, which may also be considered induced behavioral deficits, included ultrasonic vocalization, social approach behavior, and marble burying behavior, as provided and described herein. These behaviors and phenotypes are utized to determine the impact and therapeutic or risk-reduction effects of administration of IL-17a antibodies and/or inhibitor of IL-17a signaling.

Exemplary compounds for the methods and uses described herein include agents or compounds that inhibit Th17 activity, including: inhibitors of RORγt activity and/or IL-17 activity. Exemplary inhibitors of RORγt activity include: TMP778 (Skepner et al. 2014, J Immunol 192:2564-2575), SR1001 (Solt et al. 2011, Nature 472:491), SR1555 (Solt et al. 2012, ACS Chem Biol 7:1515), and SR2211 (Kumar et al. 2012, ACS Chem Biol 7:672). These and other inhibitors of RORγt activity, as well as assays for detecting/assessing RORγt activity, are described in, for example, U.S Patent Application Publication Nos. 2013/0085162, 2013/0065842 and 2007/0154487; U.S. Pat. No. 9,101,600; and WO2013/036912, WO2012/074547, WO2013/079223, WO2013/178362, WO2011/112263 (SR-9805), WO2011/112264, WO2010/049144, WO2012/027965, WO2012/028100, WO2012/100732, WO2012/100734, WO2011/107248, WO2012/139775, WO2012/064744, WO2012/106995, WO2012/147916, and WO2010/049144, the entire content of each of which is incorporated herein by reference. Other inhibitors of RORγt activity are also described in Skepner et al. (2014, J Immunol 192:2564-2575), Skepner et al. (2015, Immunology doi: 10.111/imm.12444, epub ahead of print), Nishiyama et al. (2014, Bioorganic & Medicinal Chemistry 22:2799-2808; compound 5b), Fauber et al. (2014, J Medicinal Chem 57:5871-5892), Mele et al. (2013, J Exp Med 210:2181-2190), Dhar et al. (2013, Annual Reports in Medicinal Chemistry 48:169-182), Xu et al. (2011, J Biol Chem 286:22707; ursolic acid) and Huh et al. (2011, Nature 472:486-490), the entire content of each of which is incorporated herein by reference. With respect to Fauber et al. and Dhar et al., in particular, each of the references cited therein is also incorporated herein by reference in its entirety.

Exemplary inhibitors of IL-17 activity include antibodies specific for IL-17a or the IL-17 receptor (IL-17R). In a particular embodiment, the inhibitor of IL-17 activity is a human monoclonal antibody or a humanized monoclonal antibody. Such antibodies are envisioned as being able to block IL-17R engagement by IL-17a. In a more particular embodiment, the human monoclonal antibody is brodalumab (AMG 827), which is specific for the IL-17R. In another particular embodiment, the humanized monoclonal antibody is ixekizumab (LY2439821) or secukinumab (AIN457), which are specific for IL-17a. Also envisioned for use in methods described herein are antibodies specific for the p19 subunit of IL-23 or the p40 subunit of IL-23 and IL-12. Exemplary antibodies specific for the p19 subunit of IL-23 include MK-3222 (SCH 900222), CNTO 1959, and AMG 139. Exemplary antibodies specific for the p40 subunit of IL-23 and IL-12 include Stelara (ustekinumab; CNTO 1275).

Specific agents/compounds from each of the categories listed above are available commercially as follows: brodalumab (AMG 827) and AMG 139 are available from Amgen/MedImmune; ixekizumab (LY2439821) is available from Eli Lilly; secukinumab (AIN457) is available from Novartis; MK-3222 (SCH 900222) is available from Merck; CNTO 1959 and Stelara (ustekinumab; CNTO 1275) are available from Janssen Biotech (J & J).

Also envisioned for use in methods described herein are antibodies for other Th17 cell specific cytokines, such as, but not limited to IL-17f and IL-22. Antibodies and reagents specific for Th17 specific cell surface proteins, of which CCR6 is an example, are also envisioned for use in methods described herein. See also Hedrick et al. (2010, Expert Opin Ther Targets 14:911-922), the entire content of which is incorporated herein by reference). Blocking antibodies specific for IL-23 receptor are also envisoned for use in methods described herein.

Also envisioned for use in methods described herein are antibodies for the IL-23 receptor (IL-23R). See, for example, US 2014/0275490, which is incorporated herein in its entirety by reference.

Also envisioned herein are antibody fragments or altered/mutated antibodies, particularly those wherein the Fc domain is absent or altered/mutated such that the antibody fragment or mutated antibody can no longer bind to Fc receptors. Methods for generating antibody fragments or mutated antibodies that can no longer bind to Fc receptors are described in Firan et al. (2001, Intern Immunol 13:993-1002), the entire content of which is incorporated herein by reference.

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies; Immunoglobulin molecules can be of any class (e.g., IgG, IgE, IgM, IgD, IgA, and IgY), or subclass (e.g., IgG1, IgG2, IgG3, IgG4, IgA1, and IgA2). Also included within the meaning of the term “antibody” are any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv) fragment, which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); (xii) a minibody, which is a bivalent molecule comprised of scFv fused to constant immunoglobulin domains, CH3 or CH4, wherein the constant CH3 or CH4 domains serve as dimerization domains (Olafsen T et al (2004) Prot Eng Des Sel 17(4):315-323; Hollinger P and Hudson P J (2005) Nature Biotech 23(9):1126-1136); and (xiii) other non-full length portions of heavy and/or light chains, or mutants, variants, or derivatives thereof, alone or in any combination.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

An “antibody combining site” is that structural portion of an antibody molecule comprised of light chain or heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Antibodies may also be bispecific, wherein one binding domain of the antibody is a specific binding member of the invention, and the other binding domain has a different specificity, e.g. to recruit an effector function or the like. Bispecific antibodies of the present invention include wherein one binding domain of the antibody is a specific binding member of the present invention, including a fragment thereof, and the other binding domain is a distinct antibody or fragment thereof, including that of a distinct anti-cancer or anti-tumor specific antibody. The other binding domain may be an antibody that recognizes or targets a particular cell type, as in a neural or glial cell-specific antibody. In the bispecific antibodies of the present invention the one binding domain of the antibody of the invention may be combined with other binding domains or molecules which recognize particular cell receptors and/or modulate cells in a particular fashion, as for instance an immune modulator (e.g., interleukin(s)), a growth modulator or cytokine or a toxin (e.g., ricin) or anti-mitotic or apoptotic agent or factor. Thus, the TGFbeta-1 antibodies of the invention may be utilized to direct or target agents, labels, other molecules or compounds or antibodies in indications such as wound healing, inflammation, cancer or tumors.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may also contain an antibody molecule having a plurality of antibody combining sites, each immunospecific for a different antigen; e.g., a bispecific (chimeric) monoclonal antibody.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains. Preferably, an antigen binding domain comprises an antibody light chain variable region (VL) and an antibody heavy chain variable region (VH).

Immunoconjugates or antibody fusion proteins of the present invention, wherein the antibodies, antibody molecules, or fragments thereof, of use in the present invention are conjugated or attached to other molecules or agents further include, but are not limited to such antibodies, molecules, or fragments conjugated to a chemical ablation agent, toxin, immunomodulator, cytokine, cytotoxic agent, chemotherapeutic agent, antimicrobial agent or peptide, cell wall and/or cell membrane disrupter, or drug.

One skilled in the art can readily determine or assess the suitability of other compounds for use in the invention by screening in cellular assays of Th17 activity such as those described herein or known in the art, or in animal models of disease in which Th17 cell activity is implicated such as those described herein and elsewhere. See, for example, U.S Patent Application Publication No. 2007/0154487, the entire content of which is incorporated herein by reference.

Further to the above, an MIA rhesus monkey model has also been described. See, for example, Bauman et al. (2014, Biol Psychiatry 75:332-341), which is incorporated herein by reference in its entirety. In the rhesus monkey model, pregnant rhesus monkeys in whom MIA has been induced give birth to offspring with abnormal repetitive behaviors, communication, and social interactions. Accordingly, both the MIA mouse model and the MIA rhesus monkey model provide suitable animal models in which to examine the effects of an activated maternal immune system on fetal development and the ramifications thereof in the offspring subsequently born. These animal models also provide suitable in vivo assays for evaluating potential therapeutics for administration to the pregnant females carrying fetuses who are at risk for developing a psychiatric disorder, particularly autism spectrum disease (ASD), autism and/or schizophrenia for treatment or reduction of risk or reduction of behavioral deficits and/or phenotypes of a psychiatric disorder, particularly autism spectrum disease (ASD), autism and/or schizophrenia.

Therefore, if appearing herein, the following terms shall have the definitions set out below.

The term “reduced risk of developing a psychiatric disorder” as used herein refers to a decrease in the risk that a subject will develop a psychiatric disorder.

The term “hyperinflammatory condition” as used herein refers to a condtion in a subject wherein Th17 cell activity and potentially that of related T cells, such as, for example, CD8 and γδT cell receptor (TCR) T cells with similar RORγt-dependent cytokine programs is elevated relative to a suitable control subject. With regard to pregnant females, it is understood that Th17 cell activity is elevated relative to non-pregnant females. Accordingly, a “hyperinflammatory condition” in a pregnant female induced, for example, by environmental conditions, toxins, and/or an infection (e.g., viral, bacterial, or fungal) is compared relative to that of a pregnant female that has not been exposed to the aforementioned inducers or the like.

“Preventing” or “prevention” refers to a decreased likelihood of acquiring a disease or disorder. The term may be used to encompass a decreased likelihood of at least 50%, at least 60%, at least 70%, at least 80%, at least 90%, at least 95%, at least 96%, at least 97%, at least 98%, or at least 99% relative to a control subject.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Therapeutically effective amount” means the amount of a compound that, when administered to a subject for treating a disease or disorder, is sufficient to effect such treatment for the disease or disorder. The term may also be applied with respect to the amount of a compound that, when administered to a subject for reducing the risk of developing a disease or disorder (e.g., autism spectrum disorder), is sufficient to effect such a reduction in the risk of developing the disease or disorder. The “therapeutically effective amount” can vary depending on the compound, the disease and its severity, and the age, weight, etc., of the subject to be treated.

The term ‘treating’ or ‘treatment’ of any disease or infection refers, in one embodiment, to ameliorating the disease or infection (i.e., arresting the disease or growth of the infectious agent or reducing the manifestation, extent or severity of at least one of the clinical symptoms thereof). In another embodiment ‘treating’ or ‘treatment’ refers to ameliorating at least one physical parameter of the disease. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

The agents and compounds and derivatives thereof of use in the invention may be prepared in pharmaceutical compositions, with a suitable carrier and at a strength effective for administration by various means to a subject in need thereof, such as a pregnant mother carrying a fetus at risk for developing autism. A variety of administrative techniques may be utilized, among them parenteral techniques such as subcutaneous, intravenous and intraperitoneal injections, catheterizations and the like. Average quantities of the agents and compounds and derivatives thereof may vary and in particular should be based upon the recommendations and prescription of a qualified physician or veterinarian.

The present invention further contemplates therapeutic compositions useful in practicing the therapeutic methods of this invention. A subject therapeutic composition includes, in admixture, a pharmaceutically acceptable excipient (carrier) and one or more of an agent, compound or derivative thereof, as described herein as an active ingredient.

The preparation of therapeutic compositions which contain agents, compounds, or derivatives thereof as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

An agent, compound, or derivative thereof can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic agent, compound, or derivative thereof-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or cell modulation desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and subsequent shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

In the rodent model of MIA, offspring born of pregnant mice exposed to viral infection or intra-peritoneal synthetic dsRNA (poly(I:C)) mimicking viral infection exhibit behavioral abnormalities reminiscent of ASD⁶. These abnormalities are characterized by the triad of social deficits, abnormal communication and repetitive behaviors^{7, 8}. Th17 cells are responsible for immune responses against extracellular bacteria and fungi, and their dysregulation is thought to underlie numerous inflammatory and autoimmune diseases⁹, including asthma, rheumatoid arthritis, psoriasis, inflammatory bowel disease (IBD) and multiple sclerosis. The nuclear receptor RORγt (Retinoic acid receptor-related Orphan nuclear Receptor gamma t) is expressed in several cell types in the immune system. It is a key transcriptional regulator for the development of Th17 cells as well as γδT cells and innate lymphoid cells (such as ILC3) that express Th17 cell-like cytokines, in both human and mouse^{10, 11, 12, 13}.

Several recent studies have suggested a relationship of ASD phenotypes with Th17 cells and their cytokine mediators. For example, elevated levels of serum IL-17a have been detected in a subset of autistic children^{14, 15}. Similarly, in the MIA mouse model, CD4⁺ T lymphocytes from affected offspring produced higher levels of IL-17a upon in vitro activation¹⁶. Further, Th17 cells and IL-17a have been detected in the decidua as well as in the serum during pregnancy in humans^{17, 18, 19}. Finally, IL-6, which was shown to be essential for MIA-mediated phenotypes, is known to induce Th17 cell differentiation²⁰. It is noteworthy, however, that numerous other inflammatory cytokines have been implicated in MIA. See, for example, Harvey et al. (2014, Brain Behav Immun 40:27), Washington et al. (2015, Epilepsy Behav 50:40), and Ballendine et al. (2015, Prog Neuropsychopharmacol Biol Psychiatry 57:155), the entire content of each of which is incorporated herein by reference. These references underscore the complexity of the inflammatory response manifest in MIA. The present inventors chose to investigate the potential role of Th17 cell signaling in MIA so as to explore its contribution to behavioral abnormalities in offspring.

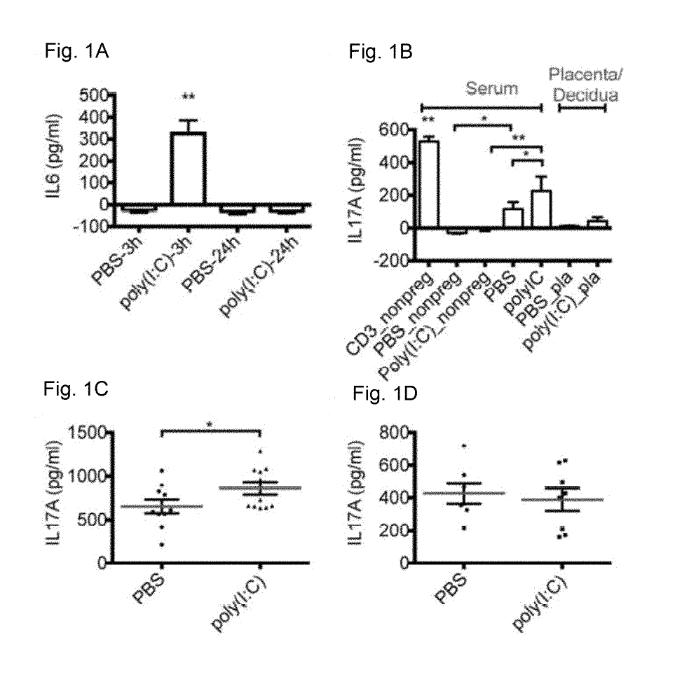

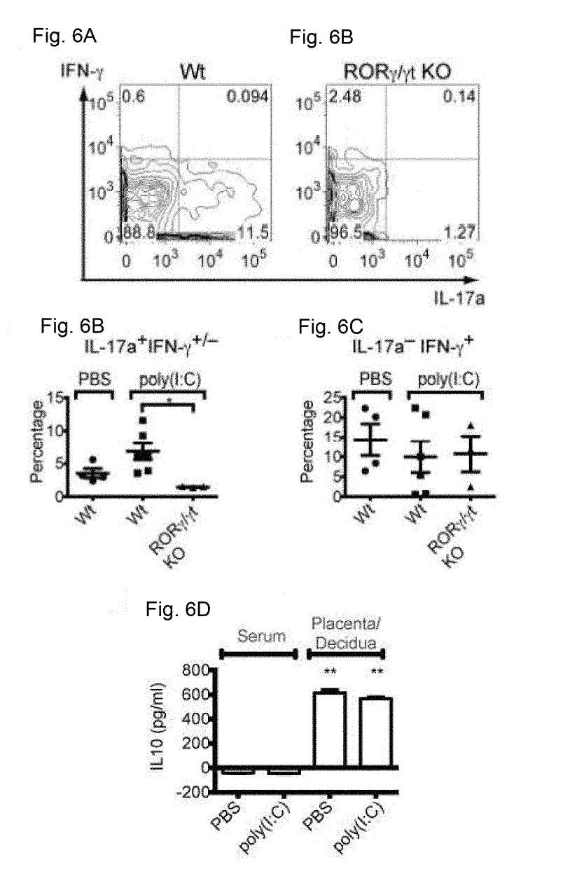

To investigate this hypothesis experimentally, the present inventors utilized the MIA mouse model. Using this model, the present inventors demonstrated that pregnant mothers injected with poly(I:C) at embryonic day 12.5 (E12.5) had strong induction of serum IL-6 levels at 3 h (FIG. 1A). In contrast, IL-17a was not detected until 2-3 days later (FIG. 1B), as would be expected if IL-6 functions upstream of IL-17a. Consistent with human data reporting pregnancy-associated increases in Th17 cells^{17, 19}, untreated or PBS-injected pregnant dams exhibited elevated serum IL-17a levels compared with non-pregnant mice (FIG. 1B). Poly(I:C)-injection resulted in a further increase of serum IL-17a at E14.5-E15.5, compared with PBS-injected dams (FIG. 1B). While IL-17a could not be detected in placenta/decidua extracts of PBS- or poly(I:C)-treated mothers (FIG. 1B), poly(I:C) treatment led to increased IL-17a secretion by placenta/decidua-associated mononuclear cells, as compared with PBS treatment (FIG. 1C and FIG. 6A-C), upon ex vivo activation with phorbolmyristate acetate (PMA) and ionomycin (mimicking T cell receptor (TCR) activation). IL-17a induction was specific to the placenta/decidua, as small intestine mononuclear cells from poly(I:C)-treated pregnant dams did not exhibit higher levels of IL-17a secretion than those from PBS-treated controls (FIG. 1D). The levels of the anti-inflammatory cytokine IL-10 exhibited an inverse pattern to that of IL-17a—it was low in the serum and high in placenta/decidua extracts—suggesting that it has an immune modulatory role at the maternal/fetal boundary (FIG. 6D). However, IL-10 levels were not affected by poly(I:C) treatment. These results, taken together, indicate that fetuses of poly(I:C)-treated pregnant females are exposed to increased levels of IL-17a, as compared with control PBS-treated animals.

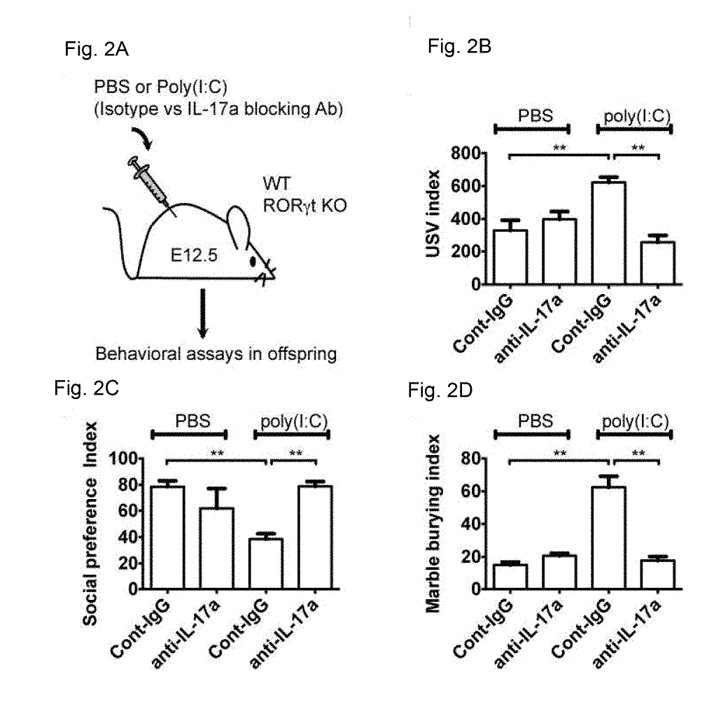

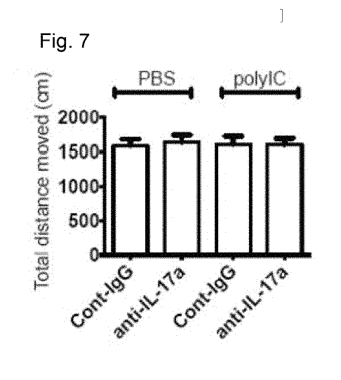

The present inventors next investigated the functional relevance of the IL-17a pathway in pregnant dams during MIA induction. Pregnant mothers were pretreated with isotype control or IL-17a blocking antibodies before injecting them with PBS or poly(I:C) (FIG. 2A) and the offspring generated thereby were subsequently assessed for abnormal communication, by measuring pup ultrasonic vocalization (USV) responses²¹. Following maternal separation, pups from poly(I:C)-injected mothers pretreated with control antibody emitted more USV calls than those from PBS-injected mothers (FIG. 2B). However, pretreating poly(I:C)-injected mothers with IL-17a blocking antibody resulted in offspring that emitted a similar number of USV calls as the pups from PBS-injected control mothers (FIG. 2B), demonstrating that IL-17a-mediated signaling events are necessary for the MIA-induced abnormal USV phenotype. As previously reported^{5, 7}, the present inventors found that prenatal exposure to MIA also caused social interaction deficits in adult offspring (FIG. 2C). Such defects were fully rescued in offspring from poly(I:C)-injected mothers pretreated with IL-17a blocking antibody (FIG. 2C). These effects were not due to group differences in total distance traveled (FIG. 7A). As repetitive behaviors are a core feature in ASD, the present inventors next sought to test repetitive behavior in mice using the marble burying assay²⁴. Offspring from poly(I:C)-injected mothers displayed enhanced marble burying compared with offspring from PBS-injected WT or HET mothers (FIG. 2D). Pretreatment with IL-17a blocking antibody of poly(I:C)-injected mothers did rescue marble burying behavior in the offspring (FIG. 2d). Taken together, these data indicate that the IL-17a pathway in pregnant mice is crucial in mediating the MIA-induced ASD-like phenotypes in offspring.

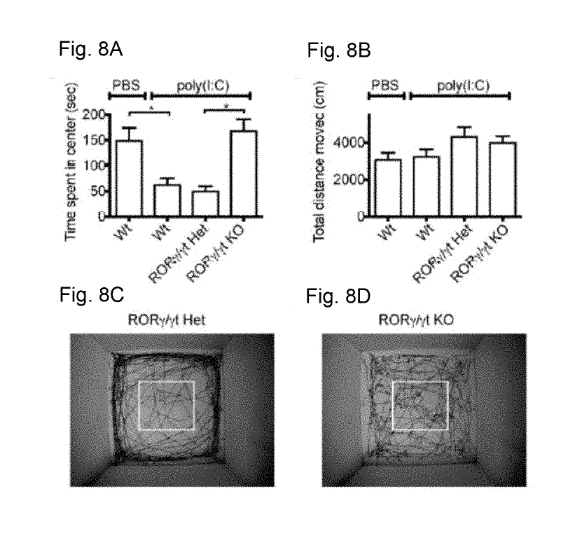

Next the present inventors investigated if maternal RORγt, like the maternal IL-17a pathway, has a role in MIA-induced behavioral phenotypes in offspring. CD45⁺ mononuclear cells, including CD4⁺ T cells, isolated from placenta and decidua of immune-activated WT mothers, but not from immune-activated mothers lacking both RORγt and the closely related RORγ isoform, produced IL-17a upon ex vivo activation with PMA and ionomycin (FIG. 6A-C). The present inventors performed an open-field test in the offspring of these mice, as MIA has been shown to induce anxiogenic behavior phenotypes in affected offspring⁵. Compared with those from PBS-injected WT control animals, offspring from poly(I:C)-injected WT or RORγ/γt HET animals spent significantly reduced time in the center of an open field, in accordance with previous studies (FIG. 8A, 8C)⁵. Importantly, MIA did not affect the total distance covered, indicating that decreased center region time was not due to a general decrease in motor activity (FIG. 8B) but to decreased exploratory behavior (i.e. increased anxiogenic behavior) in the offspring. Importantly, this behavioral change was largely absent in offspring from poly(I:C)-injected RORγ/γt KO pregnant mice (FIG. 8A, 8D). These data indicate that RORγ/γt is critical for the MIA-induced anxiogenic phenotype.

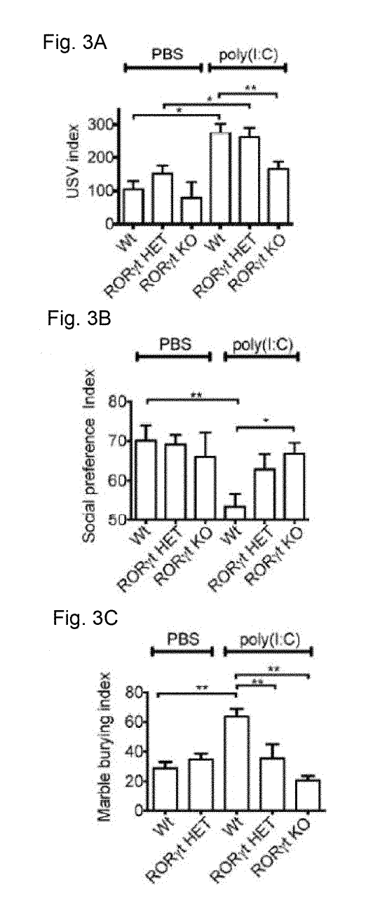

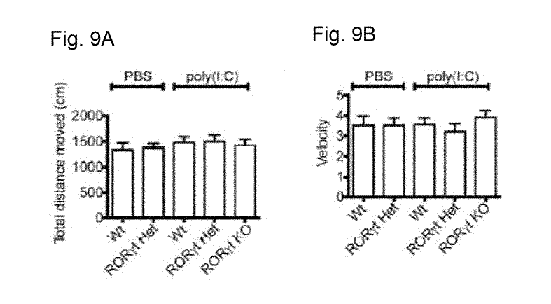

RORγ/γt-deficient mice lack RORγ/γt expression not only in CD4⁺ T cells, but also in other lymphoid and non-immune system cells, and they have defective development of secondary and tertiary lymphoid organs²⁵. To determine if RORγt functions specifically in Th17 cells to mediate MIA-induced phenotypes, the present inventors bred RORγt^{FL} animals to CD4-Cre mice to selectively inactivate Ror(c)t in the T cells of pregnant mothers (RORγt KO)²⁶. In these animals, the functions of Th17 cells (CD4⁺RORγt⁺ cells) and other RORγt-expressing αβT cells are inhibited, but RORγt-expressing innate (or innate-like) immune cells, including γδT, LTi, and ILC3^{12, 13}, as well as RORγ-expressing non-lymphoid cells are not affected. Consistent with data presented in FIG. 2, the present inventors found that prenatal exposure to MIA increased USV calls in pups derived from WT or RORγt HET mothers, and this abnormal phenotype was rescued in the offspring of RORγt KO mothers (FIG. 3A). T cell-specific deletion of maternal RORγt also rescued the MIA-induced social interaction deficit and repetitive marble burying in offspring (FIG. 3B, C). Interestingly, RORγt HET-offspring exhibited rescue of the repetitive marble burying phenotype without fully correcting abnormal USV, suggesting that MIA-induced behavioral phenotypes require different levels of RORγt activities in the affected mothers (FIG. 3A and C). These results were not due to any general mobility defects in the offspring of WT, RORγt HET, or KO mothers (FIG. 9A, B). Thus, maternal CD4⁺ T lymphocytes expressing RORγt (i.e. Th17 cells) are necessary for the induction of all three-core symptoms of ASD modeled in the offspring of MIA mothers.

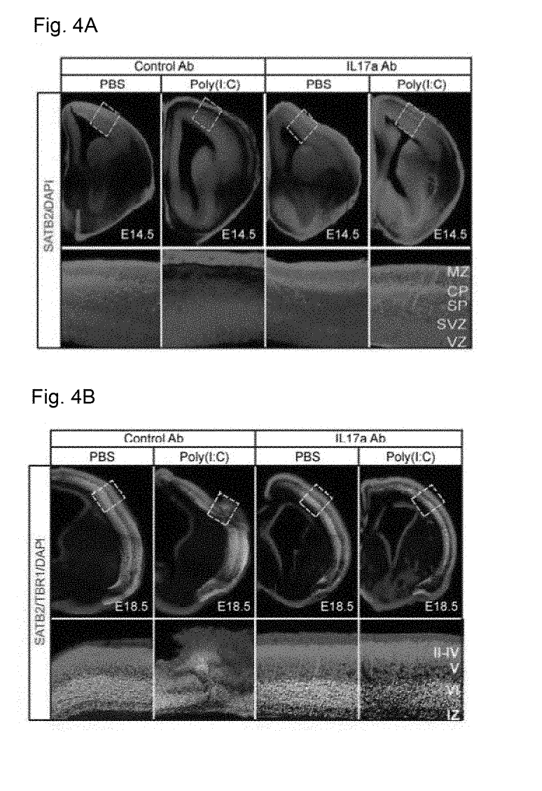

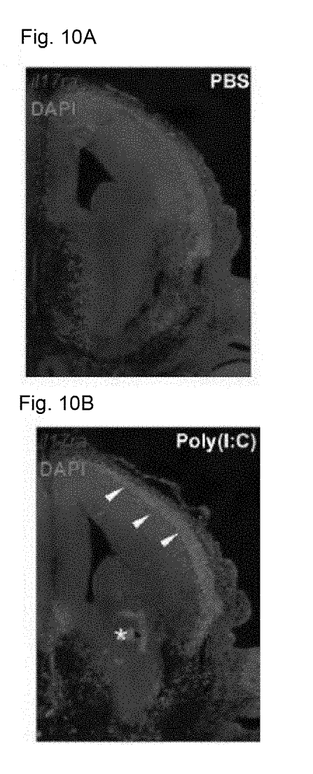

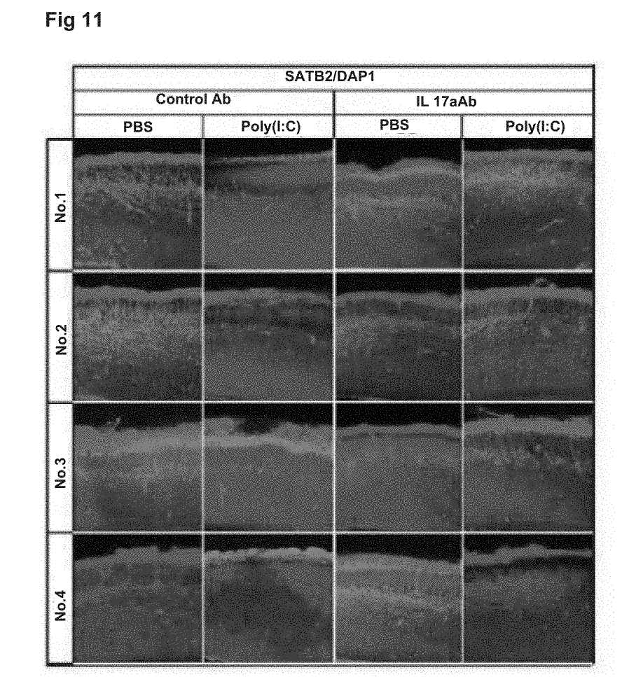

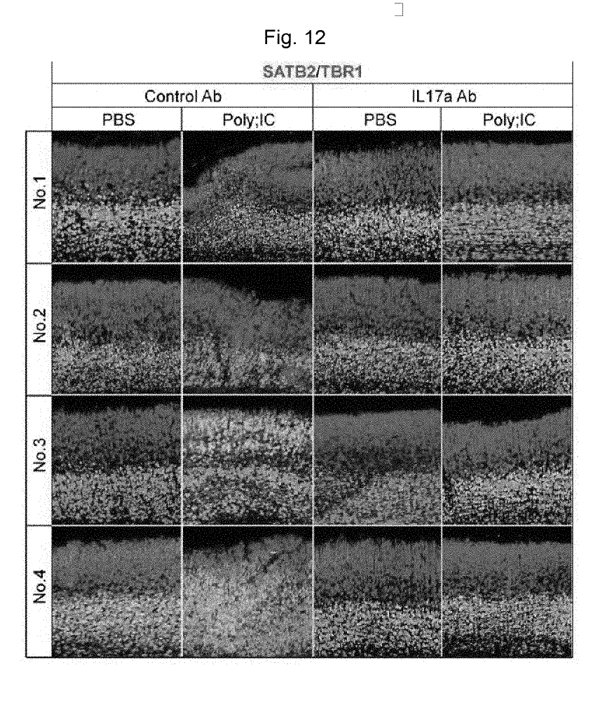

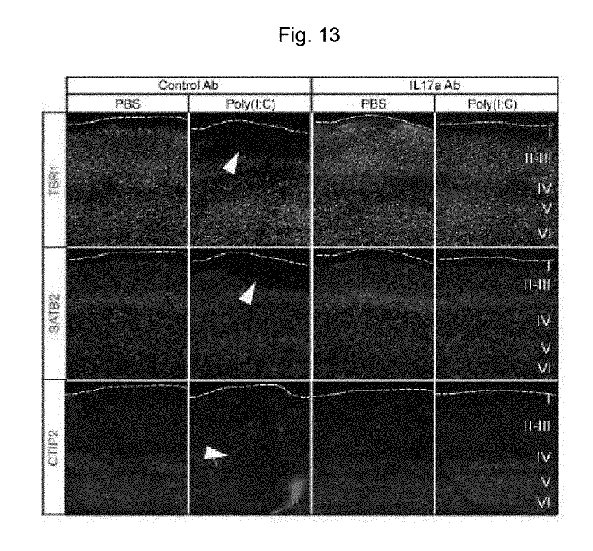

Results presented herein suggest that pathological activation of the Th17 cell/IL-17 pathway may affect fetal brain development and thereby contribute to the ASD-like behavioral phenotypes. To test this hypothesis, the present inventors chose to examine cortical development for the following reasons: 1) Cortical development starts approximately at E11 ²⁷, which correlates well with the time points of potential fetal exposure to MIA; 2) IL-17Ra expression is not only detected in the mouse cortex but also strongly up-regulated in E14.5 fetal brains following poly(I:C) injection of dams (FIG. 10A, B); and 3) Disorganized cortex or focal patches of abnormal laminar cytoarchitecture has been found in the brains of autistic patients^{28, 29}. Interestingly, Golgi staining revealed disorganized cortical regions in the offspring of MIA compared with PBS-treated mothers. The present inventors further analyzed cortical lamination in fetal brains at E14.5 and E18.5, as well as in adult brain, using antibodies specific for proteins expressed in the cortex in a layer-specific manner³⁰. MIA led to an abnormal expression of SATB2 and SATB2/TBR1 in fetal brain cortical plates at E14.5 and E18.5, respectively, compared with fetuses of control animals (FIG. 4A, B and FIGS. 11 AND 12). The abnormal expression of these markers, as well as of CTIP2, was maintained in MIA adult offspring (FIG. 13). Importantly, normal expression of SATB2/TBR1 and SATB2/TBR1/CTIP2 was largely restored in the offspring of poly(I:C)-injected WT mothers treated with IL-17a blocking antibody (FIG. 4A, B and FIGS. 11, 12 AND 13). These data demonstrate that the Th17/IL-17a-dependent pathway mediates disorganized cortical phenotypes in offspring following in utero MIA.

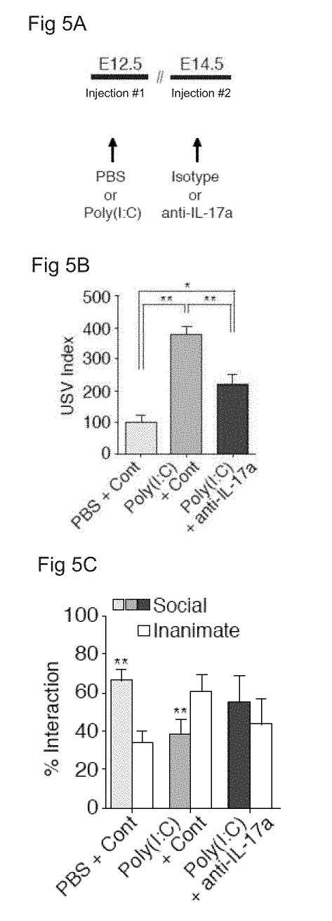

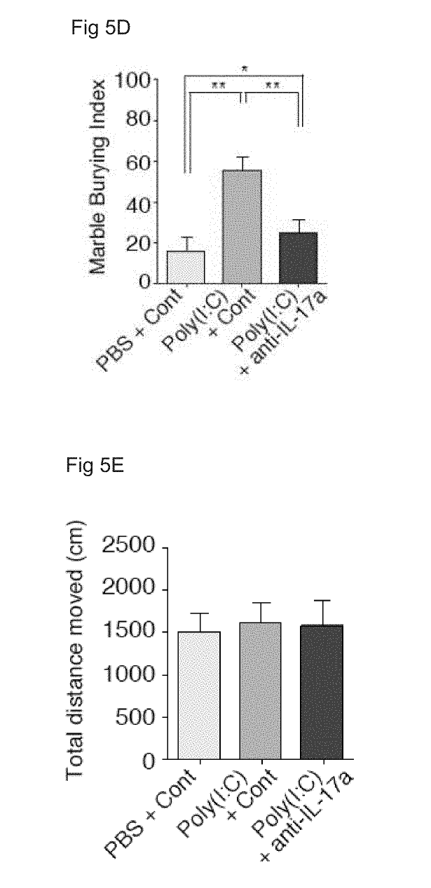

To explore further the timing parameters for administration of IL-17a blocking antibody to pregnant dams in the context of restoring normal behavior in the offspring of pregnant dams in which MIA has been induced, IL-17a blocking antibody was administered at time points after poly(I:C) injection (FIG. 5A). In that MIA-associated cortical phenotypes (as evidenced by delayed SATB2 expression and increased IL-17R expression) are observed as early as two days after poly(I:C) injection, the present inventors asked if inhibiting IL-17a after poly(I:C) injection can partially or fully rescue MIA phenotypes.

Support for the expectation that anti-IL-17a blocking antibody administered after induction of MIA phenotypes would confer benefit to offspring arises in part from the finding that IL-17a levels in the sera of poly(I:C) injected mothers continue to rise until E19.5. This evidence suggests that the enhanced levels of IL-17a at later embryonic stages may potentially be required to maintain MIA phenotypes. In keeping with this scenario, blocking IL-17a. activity in later gestational stages may ameliorate MIA phenotypes by reducing elevated IL-17a at these later stages, which in turn may be necessary for maintainence and/or reinforcement of the aberrant cortical MIA phenotypes.

Given the severity of cortical phenotypes observed at E14.5 and E18.5, however, the beneficial effects of blocking IL-17a activity may be limited or non-existent after the onset of the aberrant MIA cortical phenotypes. In keeping with this scenario, blocking IL-17a activity in later gestational stages may not be sufficient to restore MIA phenotypes, either with respect to characteristic MIA cortical developmental abnormalities or behavior in offspring of pregnant dams in whom MIA has been induced.

To address the above, the present inventors injected pregnant mothers with PBS or poly(I:C) at E12.5, followed by injection of IgG isotype control or anti-IL-17a blocking antibody at E14.5 (FIG. 5A), when the delayed expression of SABT2 manifests in MIA-exposed fetal brains (FIG. 4A). Compared to PBS injection followed by control antibody treatment, poly(I:C) injection followed by anti-IL-17a antibody administration substantially rescued USV and marble burying phenotypes (FIG. 5B and D). MIA-induced social interaction deficits were not, however, corrected (FIG. 5C). These effects were not due to group differences in mobility (FIG. 5E). These results demonstrate that treating pregnant mothers with anti-IL-17a after MIA can correct some of the ASD-like features, but pretreatment with anti-IL-17a antibody may have greater therapeutic potential.

Th17 cells are recognized as having key roles in the pathophysiology of many autoimmune diseases. Th17 cells require RORγt for their differentiation and exert their functions by secreting multiple cytokines including IL-17a. As shown herein, abrogation of RORγt expression in maternal CD4⁺ cells or blocking the IL-17 pathway in pregnant dams resulted in the complete rescue of ASD-like behavioral phenotypes in offspring of the MIA rodent model. Thus, RORγt and Th17 cells (as well as their cytokines) serve as good therapeutic targets to prevent the development of ASD phenotypes in the children of susceptible mothers. The present inventors believe that this is the first identification of a specific immune cell population that may have direct roles in inducing ASD-like phenotypes. Elucidating further downstream pathways of maternal Th17 cells, both in MIA-mothers and their offspring, will likely yield a better understanding of the mechanisms by which inflammation contributes to the development of neuropsychological disorders such as ASD.

### Methods and Materials

Animals. All experiments were performed according to the Guide for the Care and Use of Laboratory Animals and were approved by the National Institutes of Health and the Committee and Animal Care at the New York University, University of Massachusetts, University of Colorado, and Massachusetts Institute of Technology. Rorc^{Neo} and RORc(t)^{FL} mice were described elsewhere. C57BL/6 mice were obtained from Taconic (USA).

Maternal Immune Activation. Mice were mated overnight and females were checked daily for the presence of seminal plugs, noted as embryonic day 0.5 (E0.5). On E12.5, pregnant female mice were weighed and injected with a single dose (20 mg/kg; i.p.) of poly(I:C) (Sigma Aldrich) or PBS vehicle. Each dam was returned to its cage and left undisturbed until the birth of its litter. All pups remained with the mother until weaning on postnatal day 21 (P21), at which time mice were group housed at maximum 5 per cage with same-sex littermates. For the IL-17 cytokine blockade experiment, monoclonal IL-17a blocking antibody (clone 50104; R&D) or isotype control antibody (IgG2a, clone 54447; R&D) was administered 6 h before maternal immune activation (FIG. 2) via i.p. route (500 μg/animal) or 2 days after maternal immune activation (FIG. 5) via i.p. route (500 μg/animal).

Cell preparation, Flow cytometry, ELISA. Embryos at each implantation site were dissected in ice-cold HBSS containing Ca²⁺ and Mg²⁺. Myometrium was first peeled off of the decidua and embryos were discarded. Dissected decidual and placental tissues were then minced and enzymatically dissociated in HBSS containing 0.28 Wunsch units (WU)/mL Liberase (Roche) and 30 μg/ml DNase I (Roche) for 30 min at 37° C. with intermittent mixing. Digested tissues were washed in PBS containing 5 mM EDTA and 5% fetal bovine serum and then incubated again in the same buffer for 15 min at 37° C. prior to filtration through a cell strainer. After separation on a discontinuous 40% & 80% Percoll gradient, the mononuclear cell fraction was treated with ACK lysis buffer (Lonza). Mononuclear cells (1×10⁶ cells/mL) were stimulated with phorbol 12-myristate 13-acetate (PMA, 50 ng/mL; Sigma), ionomycin (500 ng/mL; Sigma) in T cell media: RPMI 1640 (Invitrogen) supplemented with 10% (v/v) heat-inactivated FBS (Hyclone), 50 U penicillin-streptomycin (Invitrogen), 2 mM glutamine, and 50 μM β-mercaptoethanol. Cell culture supernatant was used for ELISA analyses. For flow cytometry, cells were incubated for 5 h with PMA, ionomycin and GolgiStop (BD). Intracellular cytokine staining was performed according to the manufacturer's protocol (Cytofix/Cytoperm buffer set from BD with alexa647-conjugated anti-IL-17a (eBioscience) and PE-conjugated anti-IFN-γ). LSR II (BD Biosciences) and FlowJo software (Tree Star) were used for flow cytometry and analysis. Dead cells were excluded using the Live/Dead fixable aqua dead cell stain kit (Invitrogen). Placenta/decidua extract were prepared by homogenizing samples in Tissue Extraction Reagent I (Invitrogen, FN0071) in the presence of protease inhibitor cocktail (Roche, 11873580001) on ice using pestles. The homogenates were centrifuged at 4° C. at 13,000 rpm for 20 min, and the supernatants were aliquotted and frozen at −80° C. For ELISA with sera and placenta/decidua extract, IL-6 (Ebioscience), IL-17a (Biolgened), and IL-10 (BD) were measured according to the manufacturer's protocol.

Ultrasonic vocalizations. On postnatal day 9, mice were removed from the nest and placed in a 15-cm diameter dish in a sound-attenuated chamber. Mouse ultrasonic vocalizations (USVs) were detected for 3 min using an UltraSoundGateCM16/CMPA microphone (AviSoft) in the chamber under stable temperature and light control, and recorded with SAS Prolab software (AviSoft). For certain USV tests, Ultravox software (Noldus information Technology, USA) was used. An amplitude filter was used to eliminate extraneous peripheral noise (i.e. HVAC). Due to the unreliability of automated USV scoring, all pup USVs were measured and confirmed manually by observers blind to the experimental conditions.

Three-chamber social approach. Twelve-week-old male mice were tested for social behavior using a three-chamber social approach paradigm. Experimental mice were habituated for 1 h in separate clean holding cages and then introduced into a three-chamber arena with only empty object-containment cages (circular metallic cages, Stoelting Neuroscience) for a 10-min acclimation phase in two 5-min sessions in a 3-4 h period. The following day the mice were placed in the center chamber (without access to the left and right social test areas) and allowed to explore the center area for 5 min. After this exploration period, barriers to adjacent chambers were removed, allowing mice to explore the left and right arenas, which contained a social object (unfamiliar C57BL/6 male mouse) in one arena and an inanimate object (plastic toy) in the other arena. Experimental mice were given 10 min to explore both chambers and measured for approach behavior as interaction time (i.e. sniffing, approach) with targets in each chamber (within 2 cm, excluding non-nose contact or exploration). Sessions were video-recorded and object exploration time and total distance moved were analyzed using the Noldus tracking system. A social preference index was calculated as the percentage of time spent investigating the social target out of the total exploration time of both objects. Experiments were conducted with investigators blind to the treatments and genotypes of subjects. Arenas and contents were thoroughly cleaned between testing sessions. Multiple social targets from different home cages were used for testing to prevent potential odorant confounds from target home cages.

Marble burying test. One week following the social approach task, mice were acclimated for 0.5-1 h in separate clean holding cages. Mice were placed into each testing arena (arena size: 16″×8″×12″, bedding depth: 2″) containing 20 glass marbles total, which were laid out in four rows of five marbles equidistant from one another. At the end of a 15-min exploration period, mice were gently removed from the testing cages and the number of marbles buried was recorded. A marble burying index was scored as 1 for marbles covered >50% by bedding, 0.5 for ˜50% covered, or 0 for anything less.

Open field test. Adult mice were placed in a 50×50 cm white Plexiglas box. Activity was recorded by a video camera and data was analyzed with Ethovision software (Noldus). The software allows display of the paths taken by the mice, and it measures the total distance moved into the center of the arena in a 10 min session.

Immunohistochemistry. Animals were transcardially perfused with 4% paraformaldehyde in PBS under Ketamine/xylazine anesthesia. The brains were removed and sectioned at 100-μm thickness with a Leica VT1000S vibratome (Leica, USA). Slices were permeabilized with blocking solution containing 0.4% Triton X-100, 2% goat serum, and 1% BSA in PBS for 1 hr at room temperature, and then incubated with anti-TBR1 (ab31940, Abcam), anti-SATB2 (ab51502, Abcam) and anti-CTIP2 (ab18465, Abcam) antibodies overnight at 4° C. The following day, slices were incubated with fluorescently conjugated secondary antibodies (Invitrogen, USA) for 1 h at room temperature, and mounted in vectashield mounting medium with DAPI (Vector laboratories). Images of stained brain slices were acquired using a confocal microscope (LSM710; Carl Zeiss) with a 20× objective lens; all image settings were kept constant.

In Situ Hybridization. E14.5 embryos from PBS or poly(I:C)-treated mothers were collected in ice-cold PBS and subsequently fixed in 4% paraformaldehyde for 4 h at 4° C. Isolated brains were dehydrated in 30% sucrose/PBS solution overnight, and then embedded in Tissue Tek O.C.T. compound (Sakura Finetek, Torrance, Calif.). The blocks were sectioned at 16-μm thickness using a cryostat (Leica). Fluorescent in situ hybridization was performed using a branched cDNA probe with amplification technology (ViewRNA ISH Tissue Assay kit, Panomics, Santa Clara) according to the manufacturer's protocol. Briefly, the sections were rehydrated and treated with proteinase K for 20 min at 40° C., followed by re-fixation in 4% paraformaldehyde for 5 min. IL-17Ra probe were applied to the sections and incubated for 6 h at 40° C., which was designed based on the NCBI reference mRNA sequence: IL17Ra (NM 008359).

Statistics. Statistical analyses were performed using Prism. ANOVAs were followed by Holm-Sidak corrections. All data are represented as mean+/−SEM.

### Results

As described herein, the present inventors demonstrate that pro-inflammatory T cells expressing IL-17 cytokines are required in mothers for MIA to induce ASD-like phenotypes in affected offspring. Consistent with this observation, IL-17a blocking antibody administration in pregnant females protected against the development of MIA-induced behavioral deficits in the offspring. See, for example, FIG. 2. T cell-specific inactivation in mothers of RORγt, the transcription factor required for differentiation of T helper 17 (Th17) cells, a major source of IL-17a, similarly protected from induction of ASD-like phenotypes. See, for example, FIG. 3. Importantly, the present inventors also found abnormal cortical development in affected offspring, and this abnormality was rescued by inhibition of IL-17a signaling. See, for example, FIG. 4 and FIG. 13. IL-17a blocking antibody also conferred partial protection against the development of MIA-induced behavioral deficits in offspring when administered to pregnant mothers after MIA induction. See, for example, FIG. 5. These data suggest that therapeutic targeting of Th17 cells, major contributors to autoimmune disease, in susceptible pregnant mothers will reduce the likelihood of bearing children with inflammation-induced ASD phenotypes.

### REFERENCES

1. Atladottir H O, Thorsen P, Ostergaard L, Schendel D E, Lemcke S, Abdallah M, et al. Maternal infection requiring hospitalization during pregnancy and autism spectrum disorders. J Autism Dev Disord 2010, 40(12): 1423-1430.
2. Patterson P H. Immune involvement in schizophrenia and autism: etiology, pathology and animal models. Behav Brain Res 2009, 204(2): 313-321.
3. Ashwood P, Wills S, Van de Water J. The immune response in autism: a new frontier for autism research. J Leukoc Biol 2006, 80(1): 1-15.
4. Atladottir H O, Thorsen P, Ostergaard L, Schendel D E, Lemcke S, Abdallah M, et al. Maternal infection requiring hospitalization during pregnancy and autism spectrum disorders. J Autism Dev Disord 2010, 40(12): 1423-1430.
5. Smith S E P, Li J, Garbett K, Mimics K, Patterson P H. Maternal immune activation alters fetal brain development through interleukin-6. J Neurosci 2007, 27(4): 10695-10702.
6. Shi L, Fatemi S, Sidwell R, Patterson P. Maternal Influenza Infection Causes Marked Behavioral and Pharmacological Changes in the Offspring. Journal of Neuroscience 2003, 23(1): 297-302.
7. Malkova N V, Yu C Z, Hsiao E Y, Moore M J, Patterson P H. Maternal immune activation yields offspring displaying mouse versions of the three core symptoms of autism. Brain Behav Immun 2012, 26(4): 607-616.
8. Wong C C, Meaburn E L, Ronald A, Price T S, Jeffries A R, Schalkwyk L C, et al. Methylomic analysis of monozygotic twins discordant for autism spectrum disorder and related behavioural traits. Molecular psychiatry 2014, 19(4): 495-503.
9. Wilke C M, Bishop K, Fox D, Zou W. Deciphering the role of Th17 cells in human disease. Trends Immunol 2011, 32(12): 603-611.
10. Ivanov I I, McKenzie B S, Zhou L, Tadokoro C E, Lepelley A, Lafaille J J, et al. The orphan nuclear receptor RORgammat directs the differentiation program of proinflammatory IL-17+ T helper cells. Cell 2006, 126(6): 1121-1133.
11. Manel N, Unutmaz D, Littman D R. The differentiation of human T(H)-17 cells requires transforming growth factor-beta and induction of the nuclear receptor RORgammat. Nat Immunol 2008, 9(6): 641-649.
12. Spits H, Di Santo J P. The expanding family of innate lymphoid cells: regulators and effectors of immunity and tissue remodeling. Nat Immunol 2011, 12(1): 21-27.
13. Lochner M, Peduto L, Cherrier M, Sawa S, Langa F, Varona R, et al. In vivo equilibrium of proinflammatory IL-17+ and regulatory IL-10+ Foxp3+ RORgamma t+ T cells. J Exp Med 2008, 205(6): 1381-1393.
14. Al-Ayadhi L Y, Mostafa G A. Elevated serum levels of interleukin-17A in children with autism. J Neuroinflammation 2012, 9: 158-163.
15. Suzuki K, Matsuzaki H, Iwata K, Kameno Y, Shimmura C, Kawai S, et al. Plasma cytokine profiles in subjects with high-functioning autism spectrum disorders. PLoS One 2011, 6(5): e20470.
16. Mandal M, Marzouk A C, Donnelly R, Ponzio N M. Preferential development of Th17 cells in offspring of immunostimulated pregnant mice. Journal of reproductive immunology 2010, 87(1-2): 97-100.
17. Wu H X, Jin L P, Xu B, Liang S S, Li D J. Decidual stromal cells recruit Th17 cells into decidua to promote proliferation and invasion of human trophoblast cells by secreting IL-17. Cellular & molecular immunology 2014, 11(3): 253-262.
18. Nakashima A, Ito M, Yoneda S, Shiozaki A, Hidaka T, Saito S. Circulating and decidual Th17 cell levels in healthy pregnancy. Am J Reprod Immunol 2010, 63(2): 104-109.
19. Martinez-Garcia E A, Chavez-Robles B, Sanchez-Hernandez P E, Nunez-Atahualpa L, Martin-Maquez B T, Munoz-Gomez A, et al. IL-17 increased in the third trimester in healthy women with term labor. American journal of reproductive immunology 2011, 65(2): 99-103.
20. Kuchroo V K, Awasthi A. Emerging new roles of Th17 cells. European journal of immunology 2012, 42(9): 2211-2214.
21. Schwartzer J J, Careaga M, Onore C E, Rushakoff J A, Berman R F, Ashwood P. Maternal immune activation and strain specific interactions in the development of autism-like behaviors in mice. Translational psychiatry 2013, 3: e240.
22. Iwakura Y, Ishigame H, Saijo S, Nakae S. Functional specialization of interleukin-17 family members. Immunity 2011, 34(2): 149-162.
23. Esplugues E, Huber S, Gagliani N, Hauser A E, Town T, Wan Y Y, et al. Control of TH17 cells occurs in the small intestine. Nature 2011, 475(7357): 514-518.
24. Hoeffer C A, Tang W, Wong H, Santillan A, Patterson R J, Martinez L A, et al. Removal of FKBP12 enhances mTOR-Raptor interactions, LTP, memory, and perseverative/repetitive behavior. Neuron 2008, 60(5): 832-845.
25. Littman D R, Sun Z, Unutmaz D, Sunshine M J, Petrie H T, Zou Y R. Role of the nuclear hormone receptor ROR gamma in transcriptional regulation, thymocyte survival, and lymphoid organogenesis. Cold Spring Harbor symposia on quantitative biology 1999, 64: 373-381.
26. Huh J R, Leung M W, Huang P, Ryan D A, Krout M R, Malapaka R R, et al. Digoxin and its derivatives suppress TH17 cell differentiation by antagonizing RORgammat activity. Nature 2011, 472(7344): 486-490.
27. Dehay C, Kennedy H. Cell-cycle control and cortical development. Nature reviews Neuroscience 2007, 8(6): 438-450.
28. Casanova M F, El-Baz A S, Kamat S S, Dombroski B A, Khalifa F, Elnakib A, et al. Focal cortical dysplasias in autism spectrum disorders. Acta neuropathologica communications 2013, 1(1): 67.
29. Stoner R, Chow M L, Boyle M P, Sunkin S M, Mouton P R, Roy S, et al. Patches of disorganization in the neocortex of children with autism. The New England journal of medicine 2014, 370(13): 1209-1219.
30. Molyneaux B J, Arlotta P, Menezes J R, Macklis J D. Neuronal subtype specification in the cerebral cortex. Nature reviews Neuroscience 2007, 8(6): 427-437.

### EXAMPLE 2

### The Maternal Interleukin-17a Pathway in Mice Promotes Autism-Like Phenotypes in Offspring

Viral infection during pregnancy has been correlated with increased frequency of Autism Spectrum Disorder (ASD) in offspring. This observation has been modeled in rodents subjected to maternal immune activation (MIA). The immune cell populations critical in the MIA model have not been identified. Using both genetic mutants and blocking antibodies in mice, we show that RORγt-dependent effector T lymphocytes (e.g. Th17 cells) and the effector cytokine interleukin-17a (IL-17a) are required in mothers for MIA-induced behavioral abnormalities in offspring. We find that MIA induces an abnormal cortical phenotype, which is also dependent on maternal IL-17a, in the fetal brain. Our data suggest that therapeutic targeting of Th17 cells in susceptible pregnant mothers may reduce the likelihood of bearing children with inflammation-induced ASD-like phenotypes.

Several studies have suggested that viral infection of women during pregnancy correlates with an increased frequency of ASD in the offspring (1-6). In the rodent maternal immune activation model of this phenomenon (7), offspring from pregnant mice infected with virus or injected intra-peritoneally with synthetic dsRNA (poly(I:C)), a mimic of viral infection, exhibit behavioral symptoms reminiscent of ASD: social deficits, abnormal communication and repetitive behaviors (8). Th17 cells are responsible for immune responses against extracellular bacteria and fungi, and their dysregulation is thought to underlie numerous inflammatory and autoimmune diseases (9), such as asthma, rheumatoid arthritis, psoriasis, inflammatory bowel disease (IBD) and multiple sclerosis. The transcription factor Retinoic acid receptor-related Orphan nuclear Receptor gamma t (RORγt) is expressed in several cell types in the immune system. It is a key transcriptional regulator for the development of Th17 cells, as well as γδT cells and innate lymphoid cells (such as ILC3) that express Th17 cell-like cytokines, in both humans and mice (10-13).

Th17 cells and their cytokine mediators have been suggested to have a role in ASD. For example, elevated levels of IL-17a, the predominant Th17 cytokine, have been detected in the serum of a subset of autistic children (14, 15). A genome-wide copy number variant (CNV) analysis identified IL17A as one of many genes enriched in autistic patients (16). Similarly, in the MIA mouse model, CD4⁺ T lymphocytes from affected offspring produced higher levels of IL-17a upon in vitro activation (17, 18). While these data suggest that Th17 cells may be involved in ASD patients, whether Th17 cells are the specific immune cell population that is necessary for MIA phenotypes is unknown. Here we show that maternal RORγt-expressing pro-inflammatory T cells, a major source of IL-17a, are required in the MIA model for induction of ASD-like phenotypes in offspring. Consistent with this notion, antibody blockade of IL-17a activity in pregnant mice protected against the development of MIA-induced behavioral abnormalities in the offspring. Importantly, we also found atypical cortical development in affected offspring, and this abnormality was rescued by inhibition of maternal Th17/IL-17a pathways.

### Results

Elevated Fetal Brain IL-17Ra mRNA Follows Increased Maternal IL-17a in MIA

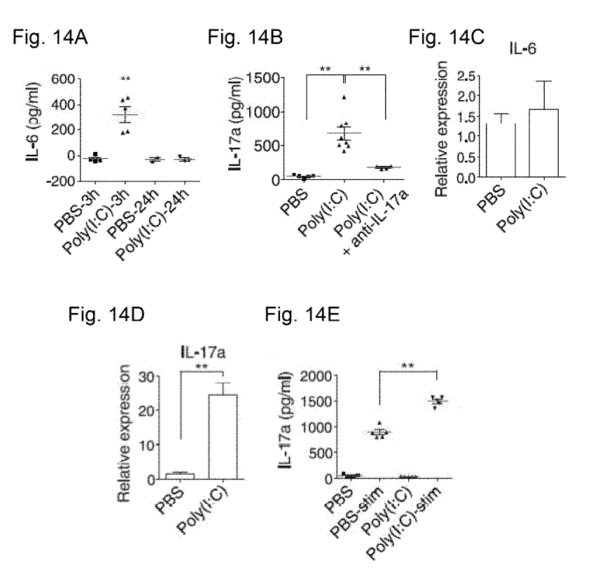

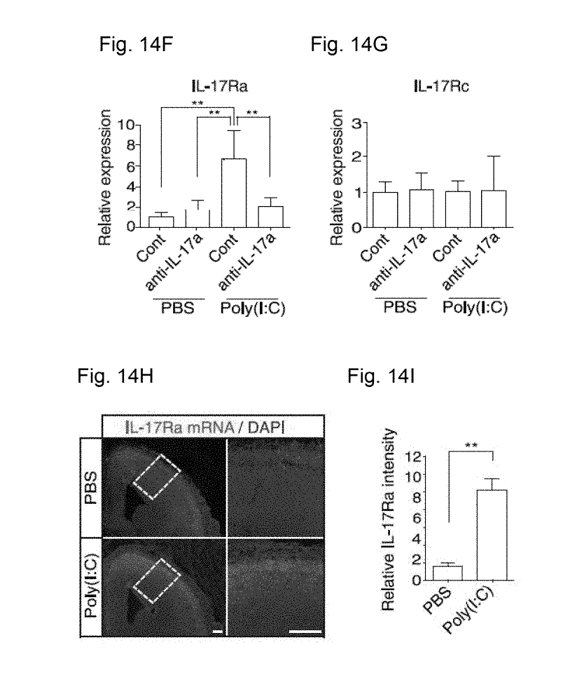

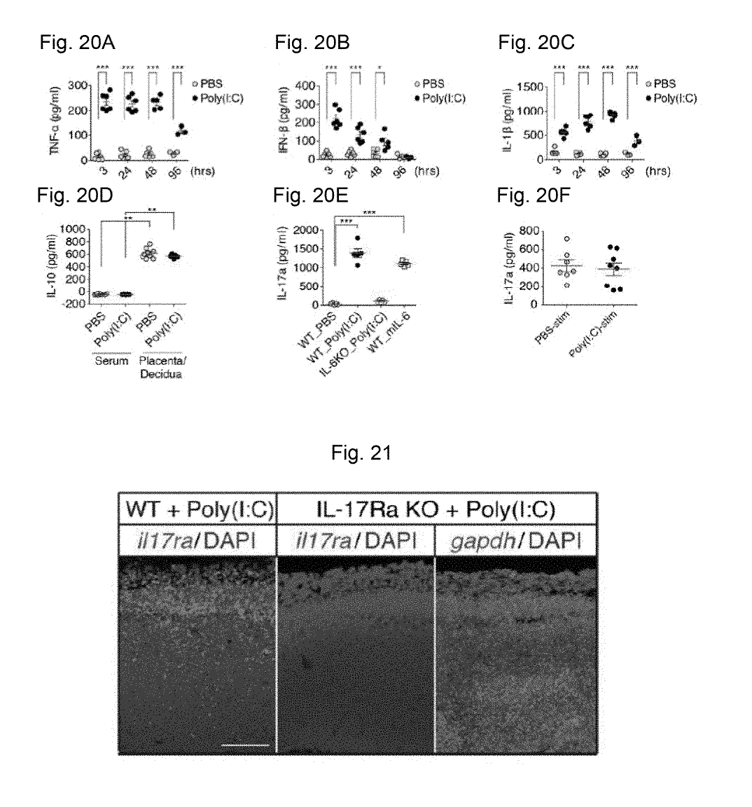

Pregnant mothers injected with poly(I:C) on embryonic day 12.5 (E12.5) had strong induction of serum cytokines IL-6, tumor necrosis factor-α (TNF-α), interferon-β (IFN-β) and IL-1β at 3 h, compared with PBS-injected control dams (FIG. 14A and 20A-C). Additionally, poly(I:C) injection resulted in a strong increase of serum IL-17a at E14.5 (FIG. 14B). On the other hand, poly(I:C) did not affect the levels of the anti-inflammatory cytokine IL-10 in the serum nor in placenta and decidua extracts (FIG. 20D). It was previously shown that the pro-inflammatory effector cytokine IL-6, a key factor for Th17 cell differentiation (19), is required in pregnant mothers for MIA to produce ASD-like phenotypes in the offspring (7). We found that poly(I:C) injection into pregnant dams lacking IL-6 (IL-6 KO) failed to increase the serum levels of IL-17a at E14.5, consistent with IL-6 acting upstream of IL-17a. Conversely, recombinant IL-6 injections into wild-type (WT) mothers were sufficient to induce IL-17a levels comparable to those of poly(I:C)-injected WT mothers (FIG. 20E). Placenta- and decidua-associated mononuclear cells, isolated from poly(I:C)-treated animals at E14.5 and cultured for 24 h, expressed similar amounts of IL-6 mRNA compared to PBS controls (FIG. 14C). In contrast, IL-17a mRNA expression in these cells was strongly up-regulated by poly(I:C) injection (FIG. 14D). This increase in mRNA expression was correlated with enhanced secretion of IL-17a by placenta- and decidua-associated mononuclear cells from poly(I:C)-treated dams (FIG. 14E), upon ex vivo stimulation with phorbolmyristate acetate (PMA) and ionomycin that mimics T cell receptor (TCR) activation. IL-17a induction was specific to the placenta and decidua, as small intestine mononuclear cells from poly(I:C)-treated pregnant dams did not secrete more IL-17a than those from PBS-treated controls (FIG. 20F).

We also observed that expression of the IL-17a receptor subunit A (IL-17Ra), but not subunit C (IL-17Rc), mRNA was strongly augmented in the fetal brain upon induction of MIA (FIG. 14F and G). By in situ hybridization, IL-17Ra mRNA was detected in the mouse cortex, and its expression was strongly up-regulated in E14.5 fetal brains following poly(I:C) injection of pregnant dams (FIG. 14H and I). The in situ probe detecting endogenous expression of IL-17Ra was specific, as it did not produce detectable signal in E14.5 fetal brain that lacks IL-17Ra (FIG. 21).

Maternal IL-17a Promotes Abnormal Cortical Development in Offspring

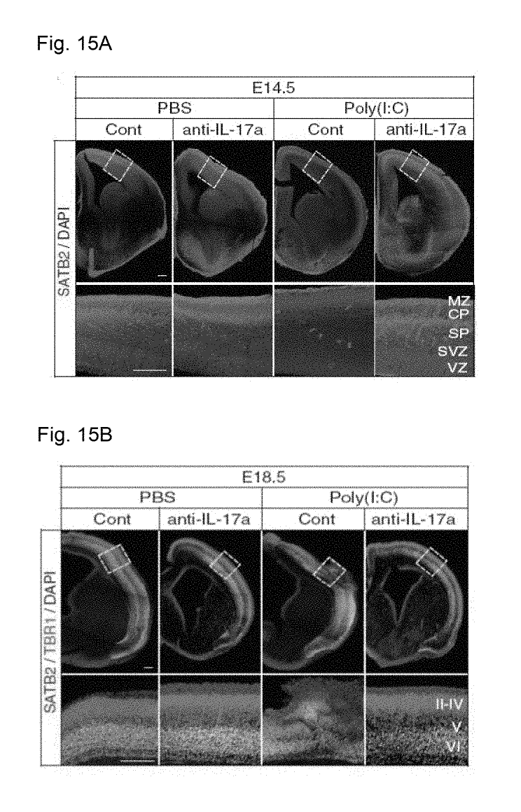

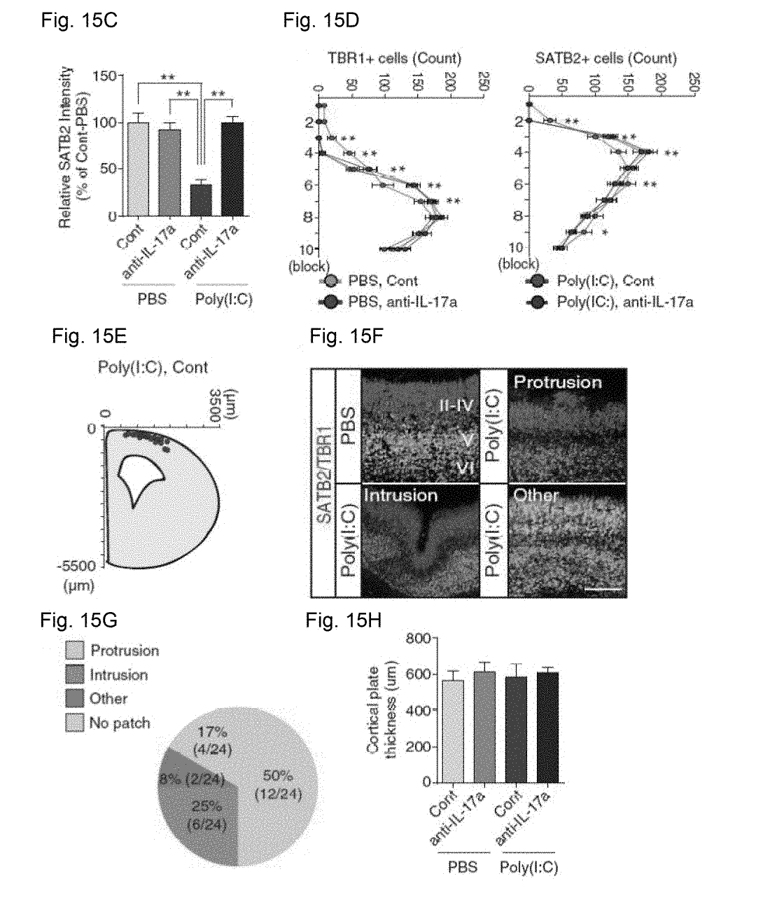

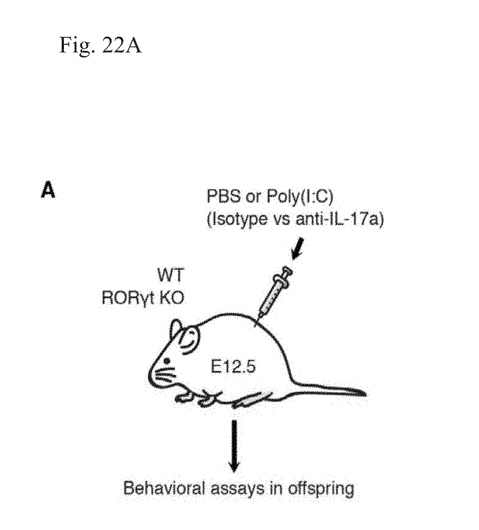

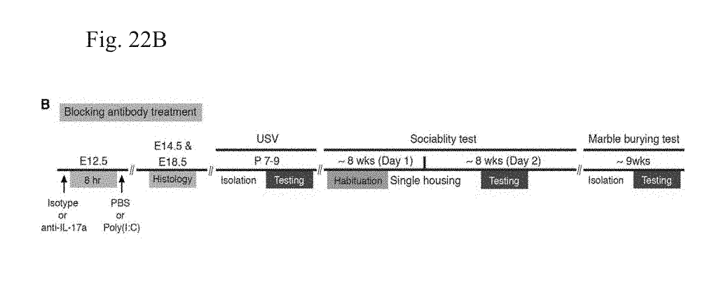

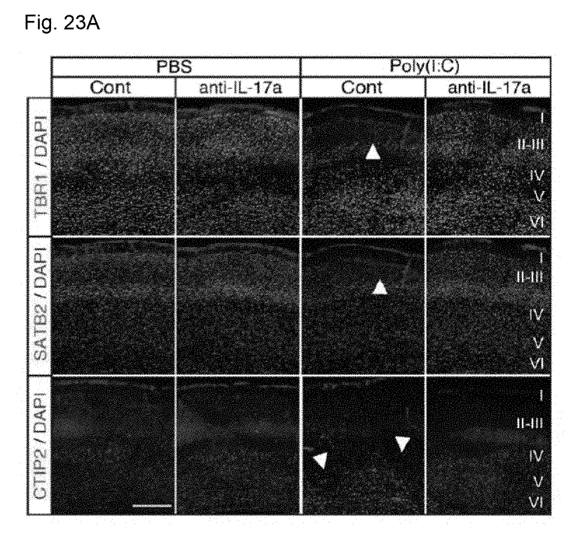

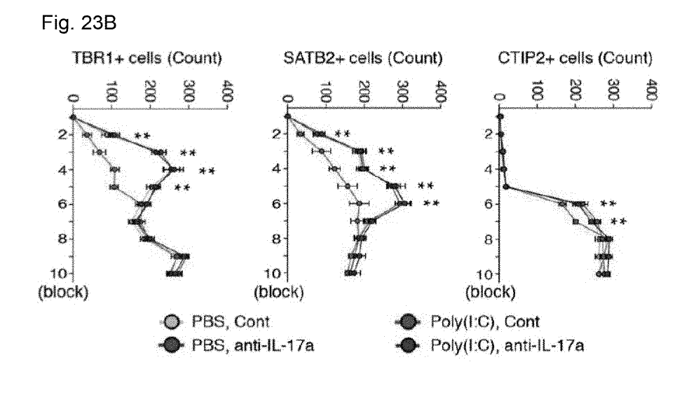

We next investigated if pathological activation of the IL-17 pathway in pregnant mothers affects fetal brain development and subsequently contributes to the ASD-like behavioral phenotypes in offspring. To test this hypothesis, we pretreated pregnant mothers with isotype control or IL-17a blocking antibodies before injecting them with PBS or poly(I:C) (FIG. 22). We then examined cortical development in the fetus for the following reasons: 1) Poly(I:C) injection of mothers increases IL-17Ra expression in the cortex of the fetal brain (FIG. 14H and I); 2) Cortical development starts approximately at Ell (20), which aligns well with the time points of potential fetal exposure to MIA (7); 3) Disorganized cortex and focal patches of abnormal laminar cytoarchitecture have been found in the brains of ASD patients (21, 22); and 4) MIA has been shown to affect cortical development (23, 24). We analyzed cortical lamination, an orderly layered structure of the developing cortex, in fetal brains at E14.5 and E18.5 as well as in the adult brain using antibodies specific for proteins expressed in the cortex in a layer-specific manner (25): Special AT-rich sequence-binding protein 2 (SATB2) (26), T-brain-1 (TBR1) (27), and chicken ovalbumin upstream promoter transcription factor-interacting protein 2 (CTIP2) (28). MIA led to delayed expression of SATB2 at E14.5 compared with fetuses of control animals (FIG. 15A, C). At E18.5, MIA resulted in a patch of disorganized cortical cytoarchitecture (FIGS. 15B, 15D-G) but did not affect cortical thickness of the fetal brains (FIG. 15H). This singular patch of disorganized cortex occurred at a similar medial-lateral position in a majority of E18.5 fetal brains (FIG. 15E and G) derived from mothers injected with poly(I:C), but not PBS. The abnormal expression patterns of SATB2, TBR1 and CTIP2 were maintained in adult MIA offspring (FIG. 23). Importantly, normal expression of these cortical layer-specific markers, as well as laminar cortical organization, were largely preserved in the offspring of poly(I:C)-injected mothers pretreated with IL-17a blocking antibody (FIG. 15A-D and FIG. 23).

Pretreatment with IL-17a blocking antibody also suppressed the MIA-mediated increase in IL-17Ra mRNA expression in fetal brain at E14.5 (FIG. 14F). This suppression was accompanied by a reduction in maternal serum IL-17a (FIG. 14B), indicating that the upregulation of IL-17Ra mRNA in fetal brains requires maternal IL-17a signaling. Of note, IL-17a antibody blockade of the IL-17a/IL-17Ra signaling pathway did not result in a concomitant increase of the serum IL-10 levels, and IL-17a mRNA expression was not detected in fetal brain at E14.5, regardless of poly(I:C) injection. Together, these data demonstrate that the maternal IL-17a-dependent pathway mediates disorganized cortical phenotypes in offspring following in utero MIA and suggest that this may be due to exposure of the fetus and its brain to increased levels of IL-17a.

Maternal IL-17a Promotes ASD-Like Behavioral Abnormalities in Offspring

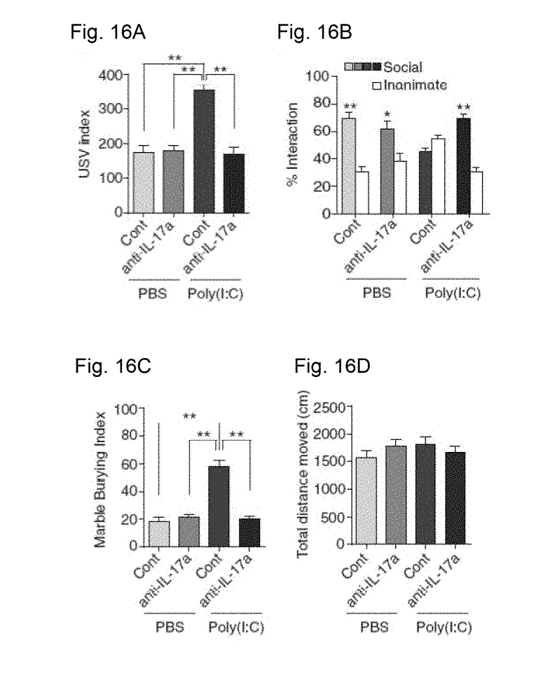

We next tested the functional relevance of the maternal IL-17a pathway for MIA-induced ASD-like behavioral abnormalities in offspring (FIG. 22). We first assessed MIA offspring for abnormal communication by measuring pup ultrasonic vocalization (USV) responses (29). Following separation from mothers, pups from poly(I:C)-injected mothers pretreated with IgG isotype control antibody emitted more USV calls than those from PBS-injected mothers (FIG. 16A), in agreement with previous studies (29, 30). Some studies have reported reduced USV calls upon MIA (8, 31), but these opposite effects may reflect differences in methodological approaches, including dose and number of exposures to poly(I:C) as well as timing of poly(I:C) administration. Altogether, these results indicate that MIA induces abnormal USV in offspring. Pretreating poly(I:C)-injected mothers with IL-17a blocking antibody resulted in offspring that emitted a similar number of USV calls as the pups from PBS-injected control mothers (FIG. 16A), demonstrating that IL-17a-mediated signaling events are necessary for the MIA-induced abnormal USV phenotype. As previously reported (7, 8), we found that prenatal exposure to MIA also caused social interaction deficits in adult offspring (FIG. 16B). This defect was fully rescued in offspring from poly(I:C)-injected mothers pretreated with IL-17a blocking antibody (FIG. 16B).

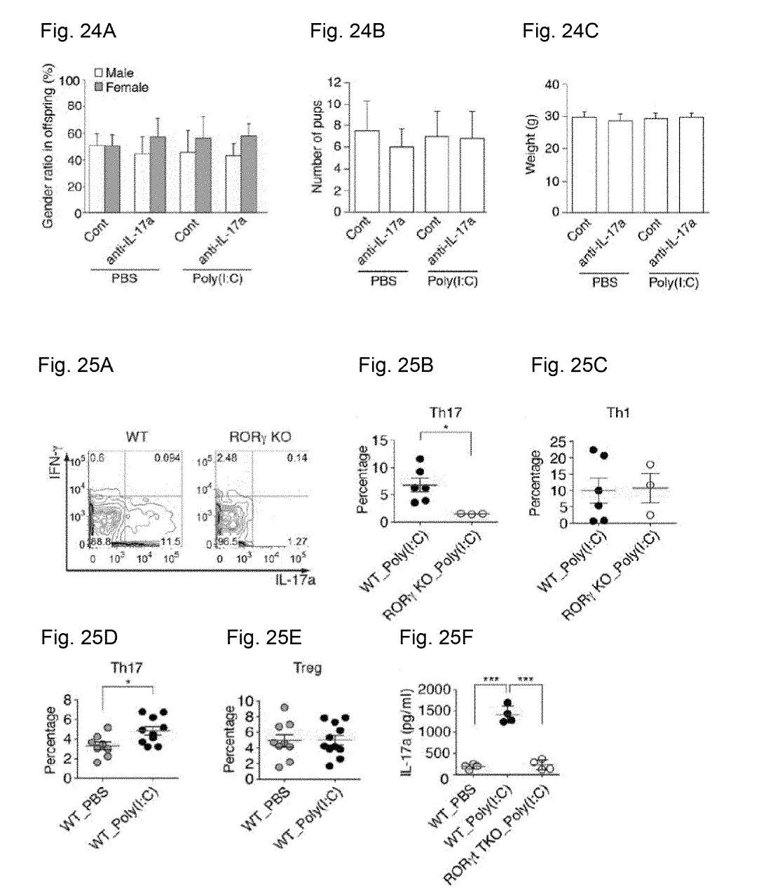

Repetitive/perseverative behaviors are another core feature in ASD that we tested next in our experimental mice using the marble burying assay (32). Offspring from poly(I:C)-injected mothers displayed enhanced marble burying compared with offspring from PBS-injected mothers (FIG. 16C), consistent with previous studies (7, 29). Pretreatment with IL-17a blocking antibody of poly(I:C)-injected mothers rescued marble burying behavior in the offspring (FIG. 16C). Importantly, distinct behavioral phenotypes observed among different treatment groups were not due to differences in activity or arousal as total distances moved during the sociability or marble burying tests were indistinguishable (FIG. 16D). Moreover, different treatment groups displayed comparable gender ratios, litter sizes, and weights (FIG. 24). Taken together, these data indicate that the IL-17a pathway in pregnant mice is crucial in mediating the MIA-induced behavioral phenotypes in offspring.

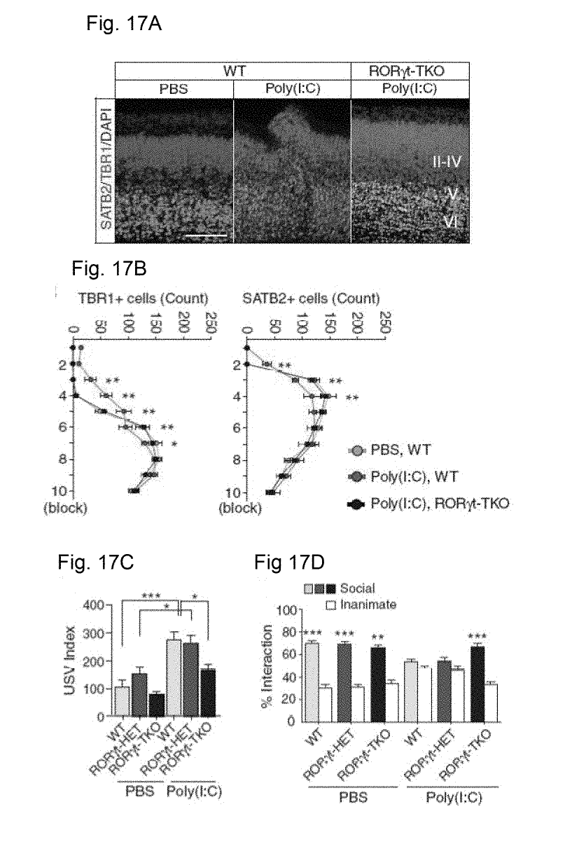

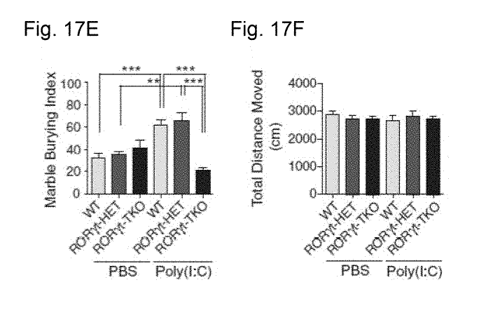

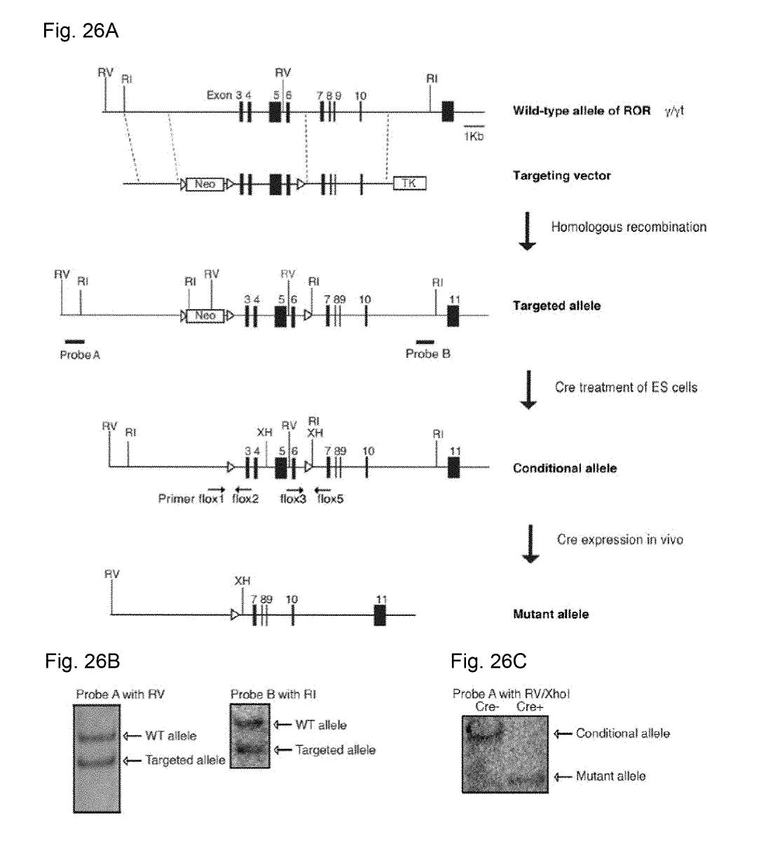

RORγt expression in maternal T cells is required for ASD-like phenotypes in the MIA offspring. As RORγt is a critical regulator of the IL-17a pathway (13), we next investigated the role of maternal RORγt in MIA-induced behavioral phenotypes in offspring. Importantly, Th17 cells and IL-17a have been detected in the decidua as well as in the serum during pregnancy in humans (33-35). CD45⁺ mononuclear cells, including CD4⁺ T cells, isolated from placenta and decidua of immune-activated WT mothers, but not from immune-activated mothers lacking both RORγt and the closely related RORγ isoform (RORγ KO), produced IL-17a upon ex vivo activation with PMA and ionomycin (FIG. 25A and B). Cells isolated from WT and RORγ KO mice secreted similar amounts of IFN-γ, consistent with the specific effect of RORγt on IL-17a expression (FIG. 25C). In line with this observation, poly(I:C) treatment increased placenta/decidua-associated Th17 but not regulatory T (Treg) cells in pregnant dams, compared with PBS treatment (FIG. 25D and E). RORγ KO mice lack RORγ/γt expression not only in CD4⁺ T cells, but also in other lymphoid and non-immune system cells, and they have defective development of secondary and tertiary lymphoid organs (36, 37). To determine if RORγt function specifically in T cells mediates MIA-induced phenotypes, we bred RORγt^{FL} animals (FIG. 26) to CD4-Cre mice to selectively inactivate rorc(t) in the T cells of pregnant mothers (RORγt TKO) (38). In these animals, the functions of Th17 cells (CD4⁺RORγt⁺ cells) and other RORγt-expressing αβT cells are inhibited, but there is no effect in RORγt-expressing innate (or innate-like) immune cells, including γδT, lymphoid tissue-inducer (LTi) cells, and innate lymphoid cells type 3 (ILC3) (11, 12), as well as in RORγ-expressing non-lymphoid cells. We found that RORγt TKO mothers failed to produce IL-17a even after poly(I:C) injection (FIG. 25F). Importantly, poly(I:C)-induced malformation of the cortex was prevented in offspring from RORγt TKO mothers (FIG. 17A and B), similar to anti-IL-17a treatment (FIG. 15B and D). Moreover, we found that prenatal exposure to MIA increased USV calls in pups derived from WT or RORγt HET mothers, but offspring of RORγt TKO mothers had normal USV behavior (FIG. 17C). T cell-specific deletion of maternal RORγt also abrogated the MIA-induced social interaction deficit and excessive marble burying in offspring (FIG. 17D and E). These results were not due to general activity defects in the offspring of WT, RORγt HET, or TKO mothers (FIG. 17F). Since these offspring were derived from mating RORγt WT/HET/TKO female with WT male mice, they all carried at least one copy of functional RORγt. Therefore, the rescue of MIA-induced phenotypes observed in the offspring of RORγt TKO mothers was not likely due to the lack of Th17 cells in the offspring. Taken together, these data indicate that maternal CD4⁺ T lymphocytes expressing RORγt (i.e. Th17 cells) are necessary for the MIA-mediated expression of cortical abnormalities and three ASD-like behaviors modeled in mouse offspring.

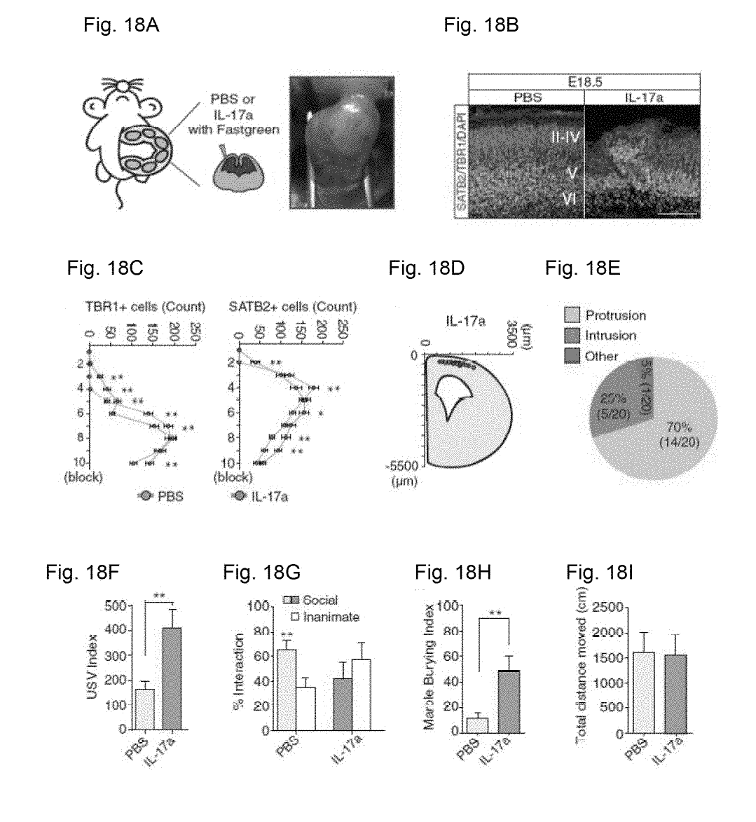

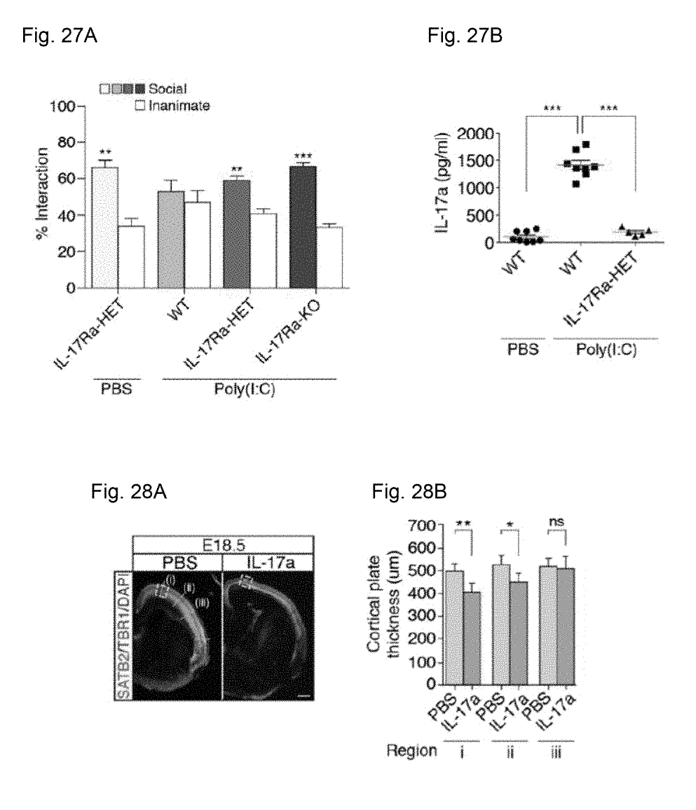

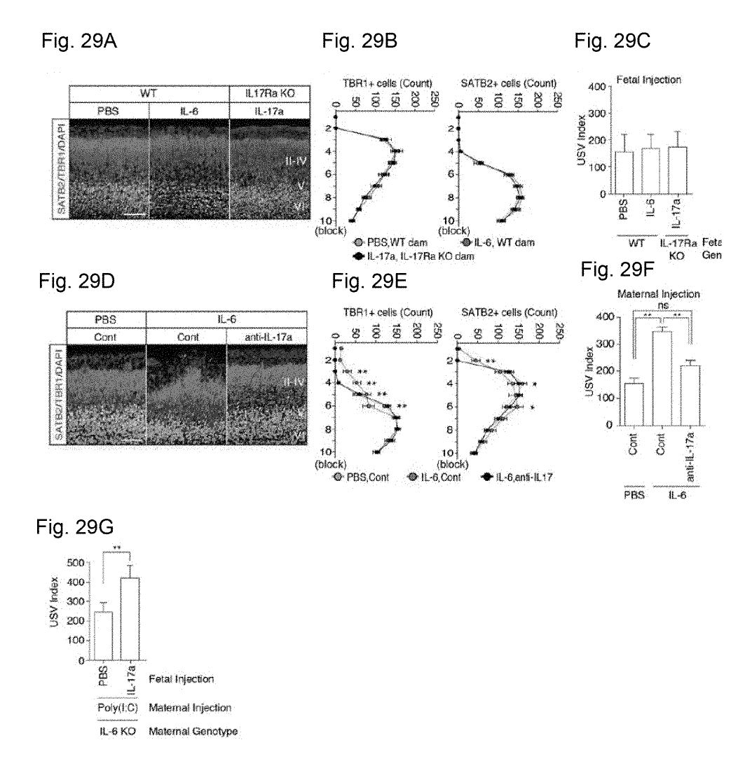

IL-17a administration to the fetal brain promotes abnormal cortical development and ASD-like behavioral phenotypes To determine if IL-17a acts on receptors in the mother or the fetus to induce the MIA phenotype, we injected poly(I:C) into IL-17Ra WT, HET or KO mothers that had been bred to IL-17Ra WT or HET males (39). Removing one or both copies of il17ra in the mother was sufficient to rescue the MIA-induced sociability deficit in offspring regardless of their genotypes (FIG. 27A). Moreover, we found that reduced expression of maternal IL-17Ra in il17ra HET mothers led to reduced serum IL-17a in poly(I:C)-treated mothers (FIG. 27B). Thus, it is difficult, if not impossible, to test the functional significance of the IL-17Ra in offpsring with a full germline il17ra KO without affecting maternal Th17 cell activity. To circumvent this problem, we asked if increasing IL-17Ra activity in the offspring, by introducing IL-17a directly into the fetal brain in the absence of maternal inflammation, would be sufficient to induce MIA phenotypes. Injection of recombinant IL-17a protein into the ventricles of the fetal brain at E14.5 in the absence of MIA (FIG. 18A) led to the appearance of disorganized cortical patches in a similar location to those induced by MIA (FIG. 18B-18E). Unlike poly(I:C) injection, however, intra-ventricular injection of IL-17a resulted in thinned cortical plates at the medial but not lateral part of the brain (FIG. 28). This effect may reflect differences in the levels or types of inflammation associated with poly(I:C) versus IL-17a injections or the time points at which poly(I:C) (E12.5) and IL-17a (E14.5) were administered. We also found that, compared with sham injection, IL-17a injections led to an enhanced USV phenotype, social approach deficit and increased marble burying behavior, all similar in magnitude to that observed in MIA-exposed offspring (FIG. 18F-H). These behavioral abnormalities were not due to group differences in mobility (FIG. 18I). Importantly, neither cortical disorganization nor enhanced USV phenotypes were observed following IL-17a injections into the ventricles of IL-17Ra KO fetuses or upon IL-6 injections into WT fetal brains (FIG. 29A-C), suggesting that IL-17a, but not IL-6, acts directly in the fetal brain to induce these phenotypes. Of note, in agreement with previous reports (7, 40), IL-6 injection into pregnant WT mothers was sufficient to produce MIA-associated behavioral (enhanced USV) and cortical phenotypes in the offspring (FIG. 29D-F). Importantly, pretreatment of pregnant mothers with anti-IL-17a blocking antibody prevented the phenotypes induced by maternal IL-6 injection (FIG. 29D-F). Lastly, IL-17a injection into brains of fetuses from poly(I:C)-injected IL-6 KO mothers was sufficient to elicit increased pup USVs compared with PBS-injected controls (FIG. 29G). These data collectively demonstrate that activation of the IL-17Ra pathway in the fetal brain, induced by intra-ventricular injection of IL-17a into the fetus or by intra-peritoneal injection of poly(I:C) or IL-6 into pregnant mothers, results in MIA-associated phenotypes in the offspring.

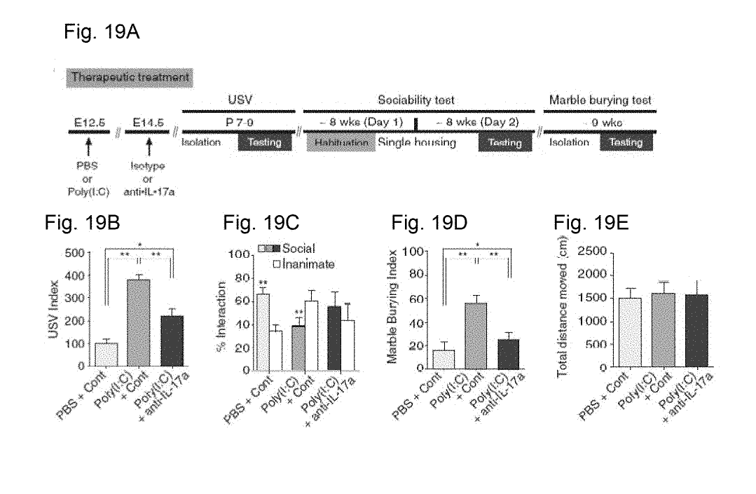

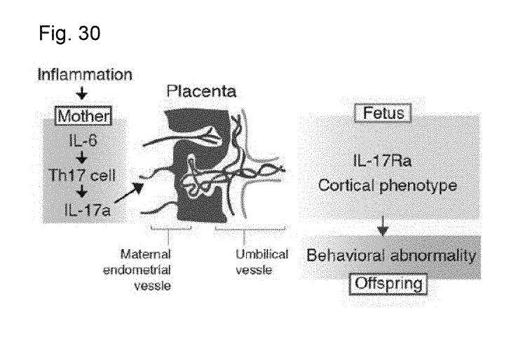

Therapeutic treatment with anti-IL-17a blocking antibody in pregnant dams ameliorates MIA-associated behavioral abnormalities Our results suggest that pathological activation of the Th17 cell/IL-17 pathway during gestation in mothers with some inflammatory conditions may alter fetal brain development and contribute to the ASD-like behavioral phenotypes in offspring (FIG. 30). Th17 cells require RORγt for their differentiation and exert their functions by secreting multiple cytokines, including IL-17a. Abrogation of RORγt expression in maternal αβT cells or blockade of the IL-17 pathway in pregnant dams resulted in the complete rescue of cortical developmental abnormalities and ASD-like behavioral phenotypes in offspring in the MIA rodent model. Thus, RORγt and Th17 cells (as well as their cytokines) may serve as good therapeutic targets to prevent the development of ASD phenotypes in the children of susceptible mothers. To further test this idea, we administered anti-IL-17a antibody to pregnant mice in a time window following MIA induction (FIG. 19). We injected pregnant mothers with PBS or poly(I:C) at E12.5, followed by injection of IgG isotype control or anti-IL-17a blocking antibody at E14.5, when the delayed expression of SABT2 manifests in MIA-exposed fetal brains (FIG. 15A and C). Compared to PBS injection followed by control antibody treatment, poly(I:C) injection followed by anti-IL-17a antibody administration partially rescued USV and marble burying phenotypes (FIG. 19B and D). However, MIA-induced social interaction deficits were not corrected (FIG. 19C). These effects were not due to group differences in mobility (FIG. 19E). Thus, treating pregnant mothers with anti-IL-17a after MIA can correct some of the ASD-like features, but pretreatment with anti-IL-17a antibody may have greater therapeutic potential.

### Conclusions

Our results identify a specific maternal immune cell population that may have direct roles in inducing ASD-like phenotypes by acting on the developing fetal brain. These findings raise the possibility that modulation of the activity of a cytokine receptor, IL-17Ra, in the central nervous system can influence neuronal development, with implications as to specification of neuronal cell types and their connectivity. Furthermore it is worthwhile to note that the loss of certain genes that induce ASD-like phenotypes were also found with defects in cortical lamination (41, 42). These observations raise the possibility that some genetic and environmental factors that have roles in the etiology of ASD function by way of similar physiological pathways. A related question is whether IL-17Ra signaling has a normal physiological function in the fetal and adult brain, especially given the structural similarities observed between the IL-17 family cytokines and neurotrophin proteins (e.g. nerve growth factor) (43, 44). Elucidating further downstream pathways of maternal IL-17a-producing T cells, both in MIA-mothers and their offspring, will likely yield a better understanding of the mechanisms by which inflammation in utero contributes to the development of neurodevelopmental disorders such as ASD and may, additionally, provide insights into the roles of cytokine receptors in the central nervous system.

### Materials and Methods

Animals All experiments were performed according to the Guide for the Care and Use of_Laboratory Animals and were approved by the National Institutes of Health and the_Committee and Animal Care at the New York University, University of Massachusetts, University of Colorado, and Massachusetts Institute of Technology. Rorc(t)^{FL} mice were generated as described in FIG. 26. il17ra^{KO} and Rorc^{Neo} mice were described elsewhere (13, 45). All C57BL/6 mice used in this study were obtained from Taconic (USA), because when these mice were obtained from different sources they often exhibited more variable immunological phenotypes (46), which may affect the penetrance and specific characteristics of the MIA phenotypes.

Generation of RORγ/γt conditional knockout mice In order to develop a conditional knockout mouse line that removes both RORγ and RORγt in a Cre-dependent manner, we generated a targeting vector, from C57BL/6-_derived BAC clone RP24-318 I7, in which two loxP sites flanked common exons 3-6. Cre-mediated deletion of exons 3-6 generates a frame shift mutation. Linearized targeting vector was then electroporated into albino C57BL/6 ES cells (CY2.4) in the gene targeting facility at the Rockefeller University. Homologous recombination was confirmed by Southern blot analyses with two different probes, as described in FIG. 26. To remove the neomycin resistance cassette, two ES cell lines with correctly targeted alleles were transiently electroporated with a Cre recombinase vector. ES cells with correct conditional alleles were confirmed by both Southern blot and PCR analyses and subsequently injected into blastocysts at the NYU gene targeting facility. For generating Southern blot probes, we used the following primers (ROR5Pr3s 5′-CCCAGCAGGTAAATCAGTGGTTC-3′ and ROR5Pr3a 5′-GCGGATAGAGCAAGGTCATTGG-3′ for Probe A; ROR3Pr3s 5′-GTAACTGTGTTTATGACTCCCTGGC-3′ and ROR3Pr3a 5′-CACTCTTTCTTGACATCTCCCCTTC-3′ for Probe B). For PCR genotyping, the following primers were used (RORgflox1 5′-TTCCTTCCTTCTTCTTGAGCAGTC-3′, RORgflox2 5′-CAGAAGAAAAGTATATGTGGCTTGTTG-3′ for WT 166bps/Floxed 226bps and RORgflox3 5′-GGTCATTTACTGGACACCCTTTCC-3′, RORgflox5 5′-GCTACACAGCAAAACCTTGTCTTGG-3′ for WT 307 bps/Floxed 384 bps).

Maternal Immune Activation Mice were mated overnight and females were checked daily for the presence of seminal plugs, noted as embryonic day 0.5 (E0.5). On E12.5, pregnant female mice were weighed and injected with a single dose (20 mg/kg; i.p.) of poly(I:C) (Sigma Aldrich) or PBS vehicle. Each dam was returned to its cage and left undisturbed until the birth of its litter. All pups remained with the mother until weaning on postnatal day 21 (P21), at which time mice were group housed at maximum 5 per cage with same-sex littermates. For the IL-17 cytokine blockade experiment, monoclonal IL-17a blocking antibody (clone 50104; R&D) or isotype control antibody (IgG2a, clone 54447; R&D) were administered 6 h before maternal immune activation via i.p. route (500 μg/animal). For IL-6 cytokine injection into pregnant dams, carrier-free recombinant mouse IL-6 (R&D) was administered as a single dose (10 μg/animal; i.p.). For testing anti-IL17a therapeutic effects, IL-17a blocking antibody or isotype control antibody (as described above) was administered 2 days after maternal immune activation (500 μg/animal; i.p.).

Cell preparation, Flow cytometry, ELISA Embryos at each implantation site were dissected in ice-cold HBSS containing Ca2+ and Mg2+ (Gibco). Myometrium was first peeled off of the decidua and embryos were discarded. Dissected decidual and placental tissues were then minced and enzymatically dissociated in HBSS containing 0.28 Wunsch units (WU)/mL Liberase (Roche) and 30 μg/mL DNase I (Roche) for 30 min at 37° C. with intermittent mixing. Digested tissues were washed in PBS containing 5 mM EDTA and 5% fetal bovine serum and then incubated again in the same buffer for 15 min at 37° C. prior to filtration through a cell strainer. After separation on a discontinuous 40% & 80% Percoll gradient, the mononuclear cell fraction was treated with ACK lysis buffer (Lonza). Mononuclear cells (1×106 cells/mL) were cultured for 24 h with or without phorbol 12-myristate 13-acetate (PMA, 50 ng/mL; Sigma) and ionomycin (500 ng/mL; Sigma) in T cell media: RPMI 1640 (Invitrogen) supplemented with 10% (v/v) heat-inactivated FBS (Hyclone), 50 U penicillin-streptomycin (Invitrogen), 2 mM glutamine, and 50 μM β-mercaptoethanol. Cell culture supernatant was used for ELISA analyses. Unstimulated cells were used to prepare total RNA for qPCR analyses. For flow cytometry, cells were incubated for 5 h with PMA, ionomycin and GolgiStop (BD). Intracellular cytokine staining was performed according to the manufacturer's protocol (Cytofix/Cytoperm buffer set from BD with Pacific Blue-conjugated CD4, FITC or PerCP-Cy5.5-conjugated CD8, APC4 Cy7-conjugated TCR-β, PE-Cy7-conjugated anti-IL-17a, PE-conjugated anti-IFN-γ, PECy7-conjugated anti-CD25 and PE-conjugated Foxp3 (eBioscience). LSR II (BD Biosciences) and FlowJo software (Tree Star) were used for flow cytometry and analysis. Dead cells were excluded using the Live/Dead fixable aqua dead cell stain kit (Invitrogen). For ELISA with sera and placenta/decidua extract, IL-6 (Ebioscience), IL-17a, TNF-α, IL-1β, IFN-β (Biolgened), and IL-10 (BD) were measured according to the manufacturer's protocol.

Ultrasonic vocalizations On postnatal day 7-9, both male and female mice were removed from the nest and habituated to the testing room for 15 minutes (separate of dam). After the habituation period, mouse pups were placed in a clean 15 cm glass pyrex high wall dish. Mouse pup ultrasonic vocalizations (USVs) were then detected for 3 min using an UltraSoundGateCM16/CMPA microphone (AviSoft) in the sound attenuation chamber under stable temperature and light control (15 lux), and recorded with SAS Prolab software (AviSoft). USVs were measured between 33-125 kHz. USVs were scored as contiguous if gaps between vocalizations were <0.02 msec. For certain USV tests, Ultravox software (Noldus information Technology, USA) was used. An amplitude filter was used to eliminate extraneous peripheral noise (i.e. HVAC). Due to the unreliability of automated USV scoring, all pup USVs were measured and confirmed manually by observers blind to the experimental conditions.

Three-chamber social approach 8-12-week-old male mice were tested for social behavior using a three-chamber social approach paradigm. Experimental mice were habituated for 1 h in separate clean holding cages and then introduced into a three-chamber arena with only empty objectcontainment cages (circular metallic cages, Stoelting Neuroscience) for a 10-min acclimation phase in two 5-min sessions in a 3-4 h period. The following day the mice were placed in the center chamber (without access to the left and right social test areas) and allowed to explore the center area for 5 min. After this exploration period, barriers to adjacent chambers were removed, allowing mice to explore the left and right arenas, which contained a social object (unfamiliar C57BL/6 male mouse) in one chamber and an inanimate object (plastic toy) in the other chamber. Experimental mice were given 10 min to explore both chambers and measured for approach behavior as interaction time (i.e. sniffing, approach) with targets in each chamber (within 2 cm, excluding non-nose contact or exploration). Sessions were video-recorded and object exploration time and total distance moved were analyzed using the Noldus tracking system. A social preference index was calculated as the percentage of time spent investigating the social target out of the total exploration time of both objects. The analysis was conducted with investigators blind to the treatments and genotypes of subjects. Arenas and contents were thoroughly cleaned between testing sessions. Multiple social targets from different home cages were used for testing to prevent potential odorant confounds from target home cages.

Marble burying test One week following the social approach task, male mice were acclimated for 0.5-1 h in separate clean holding cages. Mice were placed in a testing arena (arena size: 16″×8″×12″, bedding depth: 2″) containing 20 glass marbles, which were laid out in four rows of five marbles equidistant from one another. At the end of a 15-min exploration period, mice were gently removed from the testing cages and the number of marbles buried was recorded. A marble burying index was scored as 1 for marbles covered >50% by bedding, 0.5 for ˜50% covered, or 0 for anything less.

Intraventricular cytokine injection At E14.5, uterine horns of pregnant mice were exposed by a caudal ventral midline incision (<2 cm). Each uterine horn was exteriorized carefully and each fetus was identified. Recombinant mouse IL-17a cytokine (R&D, 0.6 μL of 2 ng/μL), IL-6 (R&D, 0.6 μL of 10 ng/μL) or saline together with the dye Fast Green (Sigma, 0.3 mg/mL) was injected (3-4 μL) into the third ventricle of each embryo by a pulled micropipette. After injection of all embryos, the uterus was replaced within the abdomen and the cavity was lavaged with warm sterile saline.

Gender genotyping Genomic DNA was extracted from tail tips of each embryo. For gender discrimination of each embryo, PCR was carried out using sry (sex-determining region of the Y chromosome) gene specific primers: 5′-ACAAGTTGGCCCAGCAGAAT-3′, and 5′-GGGATATCAACAGGCTGCCA-3′.

Immunohistochemistry Fetal brains of male embryos were dissected and fixed with 4% paraformaldehyde in PBS for 6 h at 4° C. Adult brains of male offspring were perfused and fixed with 4% paraformaldehyde in PBS for overnight at 4° C. The brains were removed and sectioned at 50-μm thickness with a Leica VT1000S vibratome (Leica, USA). Slices were permeabilized with blocking solution containing 0.4% Triton X-100, 2% goat serum, and 1% BSA in PBS for 1 h at room temperature, and then incubated with anti-TBR1 (ab31940, Abcam), anti-SATB2 (ab51502, Abcam), and anti-CTIP2 (ab18465, Abcam) antibodies overnight at 4° C. The following day, slices were incubated with fluorescently conjugated secondary antibodies (Invitrogen, USA) for 1 h at room temperature, and mounted in vectashield mounting medium with DAPI (Vector laboratories). Images of stained brain slices were acquired using a confocal microscope (LSM710; Carl Zeiss) with a 20× objective lens; all image settings were kept constant. Spatial locations of the patches were registered based on their distance from the midline of the brain. These cortical malformations were quantified using cropped images containing the malformations, or the corresponding region in WT brains. The region of interest (300×300 μm²) was divided into 10 equal laminar blocks representing different depths of the cortical plate. SATB2-, TBR1-, or CTIP1-positive cells were counted using Image J software. Signal intensity in each image was normalized relative to the total signal intensity.

Real-Time PCR Total RNA was extracted from the cerebral cortex of E14.5 fetal brain of male embryo (RNase plus mini kit, Qiagen) as well as from the decidua- and the placenta-derived mononuclear cells and reverse transcribed into cDNA using oligodT (ProtoScript first strand cDNA synthesis kit, NEB) according to the manufacturer's instructions. mRNA levels of target genes (il17ra, il17rc, il17a and il6) were quantified with a Real-Time PCR System (CFX connect Real-Time PCR, Bio-Rad) using fluorescent SYBR Green technology (Bio-Rad). Real-Time PCR was performed on 2 μL of cDNA synthesized from 200 ng of total RNA. Changes in relative gene expression normalized to gapdh or actin levels were determined using the relative threshold cycle method based on the Cont-PBS group. The detailed nucleotide sequences are shown as follows:

iIl17ra
5′-CCACTCTGTAGCACCCCAAT-3′
and
5′-CAGGCTCCGTAGTTCCTCAG-3′;
il17rc
5′-GGTACTGTCCCCAGGGGTAT-3′
and
5′- GAGGCCGGTTTTCATCTCCA-3′;
il17a
5′- CTCCAGAAGGCCCTCAGACTAC -3′
and
5′- AGCTTTCCCTCCGCATTGACACAG -3′;
il6
5′- ACACATGTTCTCTGGGAAATCGT -3′
and
5′- AAGTGCATCATCGTTGTTCATACA -3′;
actin
5′- GGCTGTATTCCCCTCCATCG -3′
and
5′- CCAGTTGGTAACAATGCCATGT -3′;
gapdh
5′-AGGTCGGTGTGAACGGATTTG-3′
and
5′-TGTAGACCATGTAGTTGAGGTCA-3′

In Situ Hybridization E14.5 male embryos from PBS or poly(I:C)-treated mothers were collected in ice-cold PBS and subsequently fixed in 4% paraformaldehyde for 4 h at 4° C. Isolated brains were dehydrated in 30% sucrose/PBS solution overnight, and then embedded in Tissue Tek O.C.T. compound (Sakura Finetek, Torrance, Calif.). The blocks were sectioned at 16-μm thickness using a cryostat (Leica). Fluorescent in situ hybridization was performed using a branched cDNA probe with amplification technology (ViewRNA ISH Tissue Assay kit, Panomics, Santa Clara) according to the manufacturer's protocol. Briefly, the sections were rehydrated and treated with proteinase K for 20 min at 40° C., followed by refixation in 4% paraformaldehyde for 5 min. IL-17Ra and Gapdh probes were applied to the sections and incubated for 6 h at 40° C. The probes were designed based on the NCBI reference mRNA sequence: il17ra (NM 008359) and gapdh (NM_008084).

Statistics Statistical analyses were performed using Prism or SPSS. ANOVAs were followed by Tukey or Holm-Sidak corrections. All data are represented as mean+/−SEM. Sample sizes were estimated using post-hoc power analyses from similar previously conducted studies (32, 47).

### FIG. 16

USV statistics: F(3,121)=48.55, p<0.0001

### Post-hoc (Tukey)

PBS, Cont-IgG vs PBS, anti-IL-17a p=0.9878
PBS, Cont-IgG vs Poly(I:C), Cont-IgG p<0.0001
PBS, Cont-IgG vs. Poly(I:C), anti-IL-17a p=0.8899
PBS, anti-IL-17a vs Poly(I:C), Cont-IgG p<0.0001
PBS, anti-IL-17a vs Poly(I:C), anti-IL-17a p=0.6938
Poly(I:C), Cont-IgG vs Poly(I:C), anti-IL-17a p<0.0001
Social Interaction statistics: F(3,62)=15.16, p<0.0001

### Social vs Inanimate (Within Group)

PBS, Cont-IgG; Social vs. PBS, Cont-IgG; Inanimate p<0.0001
PBS, anti-IL-17a; Social vs. PBS, anti-IL-17a; Inanimate p=0.0021
Poly(I:C), Cont-IgG; Social vs. Poly(I:C), Cont-IgG; Inanimate p=0.1764
Poly(I:C), anti-IL-17a; Social vs. Poly(I:C), anti-IL-17a; Inanimate p=<0.0001

### Social Interaction Across Groups (Between Groups)

Antibody blockers F(1,62)=10.48, p=0.0019, Treatment F(1,62)=6.764, p=0.0116,
Interaction F(1,62)=27.59, p<0.0001.
PBS, Cont-IgG vs Poly(I:C),Cont-IgG p<0.0001
PBS, Cont-IgG vs PBS, anti-IL17a p=0.5241
PBS, Cont-IgG vs Poly(I:C), anti-IL17a p=0.967
PBS, anti-IL-17a vs Poly(I:C), Cont-IgG p<0.001
Poly(I:C), Cont-IgG vs Poly(I:C); anti-IL-17a p<0.0001
PBS, anti-IL-17a vs Poly(I:C), anti-IL-17a p=0.2285
Marble Burying statistics: F(3,61)=62.02, p<0.0001

### Post-hoc (Tukey)

PBS, Cont-IgG vs PBS, anti-IL-17a p=0.5084
PBS, Cont-IgG vs Poly(I:C), Cont-IgG p<0.0001
PBS, Cont-IgG vs. Poly(I:C), anti-IL-17a p=0.9847
PBS, anti-IL-17a vs Poly(I:C), Cont-IgG p<0.0001
PBS, anti-IL-17a vs Poly(I:C), anti-IL-17a p=0.6691
Poly(I:C), Cont-IgG vs Poly(I:C), anti-IL-17a p<0.0001

### FIG. 17

USV statistics: F(5,97)=8.936, p<0.0001

### Post-hoc (Holm-Sidak)

WT (PBS) vs. WT (IC) p<0.001
HET (PBS) vs. HET (IC) p<0.05
KO (PBS) vs. KO (IC) p=0.062
WT (PBS) vs. HET (PBS) p=0.538
HET (PBS) vs. KO (PBS) p=0.216
KO (IC) vs. WT (IC) p=0.012
WT (PBS) vs. HET (IC) p=0.002
HET (IC) vs. KO (IC) p=0.062
KO (PBS) vs. WT (IC) p<0.001
WT (PBS) vs. KO (PBS) p=0.852
HET (IC) vs. KO (PBS) p<0.001
WT (PBS) vs. KO (IC) p=0.248
HET (PBS) vs. WT (IC) p=0.001
HET (PBS) vs. KO (IC) p=0.876
HET (IC) vs. WT (IC) p=0.876
Social Interaction statistics: F(5,117)=6.904, p<0.0001

### Social vs Inanimate (Within Group)

WT-PBS p<0.0001
WT-IC p>0.9999
HET-PBS p<0.0001
HET-IC p>0.9999
KO-PBS p=0.0001
KO-IC p<0.0001

### Social Interaction Across Groups (Between Groups)

Genotype F(2,117)=1.1547, p=0.2172, Treatment F(1,117)=15.27, p=0.0002, Interaction F(2,117)=4.842, p=0.0095.
WT (PBS) vs. WT (IC) p=0.0004
HET (PBS) vs. HET (IC) p=0.0359
KO (PBS) vs. KO (IC) p=0.9999
WT (PBS) vs. HET (PBS) p>0.9999
HET (PBS) vs. KO (PBS) p=0.9822
HET (PBS) vs KO (IC) p=0.9961
WT(IC) vs. HET (IC) p=0.9999
KO (IC) vs. WT (IC) p=0.0049
WT (PBS) vs. HET (IC) p=0.0139
HET (IC) vs. KO (IC) p=0.0714
KO (PBS) vs. WT (IC) p=0.0381
WT (PBS) vs. KO (PBS) p=0.9607
HET (IC) vs. KO (PBS) p=0.1929
WT (PBS) vs. KO (IC) p=0.9878
HET (PBS) vs. WT (IC) p=0.0029

### Distance Moved (Between Groups)

Genotype F(2,113)=0.2697, p=0.7641, Treatment F(1,113)=0.6454, p=0.4234, Interaction F(2,113)=0.054, p=0.9476.
WT (PBS) vs. WT (IC) p=0.9677
HET (PBS) vs. HET (IC) p>0.9999
KO (PBS) vs. KO (IC) p=0.9980
WT (PBS) vs. HET (PBS) p=0.9819
HET (PBS) vs. KO (PBS) p=0.9988
HET (PBS) vs KO (IC) p>0.9999
WT (IC) vs. HET (IC) p=0.9996
KO (IC) vs. WT (IC) p>0.9999
WT (PBS) vs. HET (IC) p=0.720
HET (IC) vs. KO (IC) p=0.9999
KO (PBS) vs. WT (IC) p=0.9983
WT (PBS) vs. KO (PBS) p=0.9997
HET (IC) vs. KO (PBS) p=0.9893
WT (PBS) vs. KO (IC) p=0.9722
HET (PBS) vs. WT (IC) p>0.9999
Marble Burying statistics: F(5,114)=13.90, p<0.0001, Genotype F(2,114)=7.542, p<0.0001, Treatment F(1,114)=9.598, p=0.0025, Interaction F(2,114)=16.40, p<0.0001.

### Post-hoc (Tukey, Corrects for Multiple Comparisons)

WT (PBS) vs. WT (IC) p=0.0008
HET (PBS) vs. HET (IC) p=0.0015
KO (PBS) vs. KO (IC) p=0.0507
WT (PBS) vs. HET (PBS) p=0.9996
HET (PBS) vs. KO (PBS) p=0.9388
HET (PBS) vs. KO (IC) p=0.3196
WT (IC) vs HET (IC) p=0.9963
KO (IC) vs. WT (IC) p<0.0001
WT (PBS) vs. HET (IC) p=0.0015
HET (IC) vs. KO (IC) p<0.0001
KO (PBS) vs. WT (IC) p=0.0494
WT (PBS) vs. KO (PBS) p=0.8569
HET (IC) vs. KO (PBS) p=0.0483
WT (PBS) vs. KO (IC) p=0.6361
HET (PBS) vs. WT (IC) p=0.0006

### FIG. 18

USV statistics: the Student's t-test
PBS vs. IL17a p<0.0001
Social Interaction statistics: F(1,28)=28.65, p<0.0001

### Social vs Inanimate (Within Group)

PBS; Social vs. PBS; Inanimate p=0.0002
IL-17a; Social vs. IL-17a; Inanimate p=0.015
Marble Burying statistics: the Student's t-test
PBS vs. IL17a p<0.0001

### FIG. 19

USV statistics: F(2,58)=97.05, p<0.0001

### Post-hoc (Tukey)

PBS, Cont-IgG vs Poly(I:C), Cont-IgG p<0.0001
PBS, Cont-IgG vs. Poly(I:C), anti-IL-17a p<0.0001
Poly(I:C), Cont-IgG vs Poly(I:C), anti-IL-17a p<0.0001
Social Interaction statistics: F(2,36)=21.62, p<0.0001

### Social vs Inanimate (Within Group)

PBS, Cont-IgG; Social vs. PBS, Cont-IgG; Inanimate p<0.0001
Poly(I:C), Cont-IgG; Social vs. Poly(I:C), Cont-IgG; Inanimate p=0.0064
Poly(I:C), anti-IL-17a;Social vs. Poly(I:C), anti-IL-17a; Inanimate p=0.0255
Marble Burying statistics: F(2,36)=120.5, p<0.0001

### Post-hoc (Tukey)

PBS, Cont-IgG vs Poly(I:C), Cont-IgG p<0.0001
PBS, Cont-IgG vs. Poly(I:C), anti-IL-17a p=0.0121
Poly(I:C), Cont-IgG vs Poly(I:C), anti-IL-17a p<0.0001

### FIG. 27

Social Interaction statistics:

### Social vs Inanimate (Within Group)

HET-PBS t(9)=3.858, p=0.004
WT-IC t(6)=0.450, p=0.669
HET-IC t(23)=3.622, p=0.001
KO-IC t(27)=8.573, p<0.001

### Social Interaction (Between Groups)

F(3,65)=3.544, p=0.019; Genotype F(2,68)=4.848, p=0.011, Treatment F(1,69)=2.305, p=0.134,
HET (PBS) vs. WT (IC) p=0.135
HET (PBS) vs. HET (IC) p=0.433
HET (PBS) vs. KO (IC) p=0.998
WT (IC) vs. HET (IC) p=0.636
WT (IC) vs. KO (IC) p=0.042
HET (IC) vs KO (IC) p=0.113

### FIG. 29

USV statistics: the Student's t-test
PBS vs. IL17a p=0.0002
USV statistics: F(2,44)=24.59, p<0.0001

### Post-hoc (Tukey)

PBS, Cont-IgG vs IL-6, Cont-IgG p<0.0001
PBS, Cont-IgG vs IL-6, anti-IL-17a p=0.0741
IL-6, Cont-IgG vs. IL-6, anti-IL-17a p<0.0001

### REFERENCES

1. H. O. Atladottir et al., Maternal infection requiring hospitalization during pregnancy and autism spectrum disorders. J Autism Dev Disord 40, 1423-1430 (2010).
2. P. H. Patterson, Immune involvement in schizophrenia and autism: etiology, pathology and animal models. Behav Brain Res 204, 313-321 (2009).
3. A. S. Brown et al., Elevated maternal C-reactive protein and autism in a national birth cohort. Molecular psychiatry 19, 259-264 (2014).
4. H. O. Atladottir et al., Association of family history of autoimmune diseases and autism spectrum disorders. Pediatrics 124, 687-694 (2009).
5. P. Ashwood, S. Wills, J. Van de Water, The immune response in autism: a new frontier for autism research. Journal of leukocyte biology 80, 1-15 (2006).
6. B. K. Lee et al., Maternal hospitalization with infection during pregnancy and risk of autism spectrum disorders. Brain Behav Immun 44, 100-105 (2015).
7. S. E. P. Smith, J. Li, K. Garbett, K. Mimics, P. H. Patterson, in The Journal of neuroscience: the official journal of the Society for Neuroscience. (2007), vol. 27, pp. 10695-10702.
8. N. V. Malkova, C. Z. Yu, E. Y. Hsiao, M. J. Moore, P. H. Patterson, Maternal immune activation yields offspring displaying mouse versions of the three core symptoms of autism. Brain Behav Immun 26, 607-616 (2012).
9. C. M. Wilke, K. Bishop, D. Fox, W. Zou, Deciphering the role of Th17 cells in human disease. Trends Immunol 32, 603-611 (2011).
10. N. Manel, D. Unutmaz, D. R. Littman, The differentiation of human T(H)-17 cells requires transforming growth factor-beta and induction of the nuclear receptor RORgammat. Nat Immunol 9, 641-649 (2008).
11. H. Spits, J. P. Di Santo, The expanding family of innate lymphoid cells: regulators and effectors of immunity and tissue remodeling. Nat Immunol 12, 21-27 (2011).
12. M. Lochner et al., In vivo equilibrium of proinflammatory IL-17+ and regulatory IL-10+ Foxp3+ RORgamma t+ T cells. J Exp Med 205, 1381-1393 (2008).
13. I. Ivanov et al., in Cell. (2006), vol. 126, pp. 1121-1133.
14. L. Y. Al-Ayadhi, G. A. Mostafa, Elevated serum levels of interleukin-17A in children with autism. J Neuroinflammation 9, 158 (2012).
15. K. Suzuki et al., Plasma cytokine profiles in subjects with high-functioning autism spectrum disorders. PLoS One 6, e20470 (2011).
16. B. van der Zwaag et al., Gene-network analysis identifies susceptibility genes related to glycobiology in autism. PLoS One 4, e5324 (2009).
17. M. Mandal, A. C. Marzouk, R. Donnelly, N. M. Ponzio, in Journal of reproductive immunology. (2010), vol. 87, pp. 97-100.
18. E. Y. Hsiao, S. W. McBride, J. Chow, S. K. Mazmanian, P. H. Patterson, Modeling an autism risk factor in mice leads to permanent immune dysregulation. Proc Natl Acad Sci USA 109, 12776-12781 (2012).
19. V. K. Kuchroo, A. Awasthi, Emerging new roles of Th17 cells. European journal of immunology 42, 2211-2214 (2012).
20. C. Dehay, H. Kennedy, Cell-cycle control and cortical development. Nature reviews. Neuroscience 8, 438-450 (2007).
21. M. F. Casanova et al., Focal cortical dysplasias in autism spectrum disorders. Acta neuropathologica communications 1, 67 (2013).
22. R. Stoner et al., Patches of disorganization in the neocortex of children with autism. The New England journal of medicine 370, 1209-1219 (2014).
23. J. De Miranda et al., Induction of Toll-like receptor 3-mediated immunity during gestation inhibits cortical neurogenesis and causes behavioral disturbances. mBio 1, (2010).
24. S. E. Smith, R. M. Elliott, M. P. Anderson, Maternal immune activation increases neonatal mouse cortex thickness and cell density. Journal of neuroimmune pharmacology: the official journal of the Society on NeuroImmune Pharmacology 7, 529-532 (2012).
25. B. J. Molyneaux, P. Arlotta, J. R. Menezes, J. D. Macklis, Neuronal subtype specification in the cerebral cortex. Nature reviews. Neuroscience 8, 427-437 (2007).
26. E. A. Alcamo et al., Satb2 regulates callosal projection neuron identity in the developing cerebral cortex. Neuron 57, 364-377 (2008).
27. C. Englund et al., Pax6, Tbr2, and Tbrl are expressed sequentially by radial glia, intermediate progenitor cells, and postmitotic neurons in developing neocortex. The Journal of neuroscience: the official journal of the Society for Neuroscience 25, 247-251 (2005).
28. M. Leid et al., CTIP1 and CTIP2 are differentially expressed during mouse embryogenesis. Gene expression patterns: GEP 4, 733-739 (2004).
29. J. J. Schwartzer et al., Maternal immune activation and strain specific interactions in the development of autism-like behaviors in mice. Translational psychiatry 3, e240 (2013).
30. N. Yee, R. K. Schwarting, E. Fuchs, M. Wohr, Increased affective ultrasonic communication during fear learning in adult male rats exposed to maternal immune activation. Journal of psychiatric research 46, 1199-1205 (2012).
31. E. Y. Hsiao et al., Microbiota modulate behavioral and physiological abnormalities associated with neurodevelopmental disorders. Cell 155, 1451-1463 (2013).
32. C. A. Hoeffer et al., Removal of FKBP12 enhances mTOR-Raptor interactions, LTP, memory, and perseverative/repetitive behavior. Neuron 60, 832-845 (2008).
33. H. X. Wu, L. P. Jin, B. Xu, S. S. Liang, D. J. Li, Decidual stromal cells recruit Th17 cells into decidua to promote proliferation and invasion of human trophoblast cells by secreting IL-17. Cellular & molecular immunology 11, 253-262 (2014).
34. A. Nakashima et al., in American journal of reproductive immunology. (2010), vol. 63, pp. 104-109.
35. E. A. Martinez-Garcia et al., IL-17 increased in the third trimester in healthy women with term labor. American journal of reproductive immunology 65, 99-103 (2011).
36. G. Eberl, D. R. Littman, Thymic origin of intestinal alphabeta T cells revealed by fate mapping of RORgammat+ cells. Science 305, 248-251 (2004).
37. Z. Sun et al., Requirement for RORgamma in thymocyte survival and lymphoid organ development. Science 288, 2369-2373 (2000).
38. J. R. Huh et al., Digoxin and its derivatives suppress TH17 cell differentiation by antagonizing RORgammat activity. Nature 472, 486-490 (2011).
39. Y. Iwakura, H. Ishigame, S. Saijo, S. Nakae, Functional specialization of interleukin-17 family members. Immunity 34, 149-162 (2011).
40. E. Y. Hsiao, P. H. Patterson, Activation of the maternal immune system induces endocrine changes in the placenta via IL-6. Brain Behav Immun 25, 604-615 (2011).
41. L. A. Orosco et al., Loss of Wdfy3 in mice alters cerebral cortical neurogenesis reflecting aspects of the autism pathology. Nat Commun 5, 4692 (2014).
42. O. Penagarikano et al., Absence of CNTNAP2 leads to epilepsy, neuronal migration abnormalities, and core autism-related deficits. Cell 147, 235-246 (2011).
43. S. G. Hymowitz et al., IL-17s adopt a cystine knot fold: structure and activity of a novel cytokine, IL-17F, and implications for receptor binding. EMBO J 20, 5332-5341 (2001).
44. X. Zhang, P. Angkasekwinai, C. Dong, H. Tang, Structure and function of interleukin-17 family cytokines. Protein Cell 2, 26-40 (2011).
45. K. Chen et al., Th17 cells mediate clade-specific, serotype-independent mucosal immunity. Immunity 35, 997-1009 (2011).
46. Ivanov, II et al., Induction of intestinal Th17 cells by segmented filamentous bacteria. Cell 139, 485-498 (2009).
47. C. A. Hoeffer et al., Regulator of calcineurin 1 modulates expression of innate anxiety and anxiogenic responses to selective serotonin reuptake inhibitor treatment. The Journal of neuroscience: the official journal of the Society for Neuroscience 33, 16930-16944 (2013).

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

What is claimed is:

1. A method for reducing the risk of developing a psychiatric disorder in a fetus, the method comprising administering an inhibitor of T helper 17 (Th17) cell activity to a female during a pregnancy wherein the female is carrying the fetus in utero.

2. The method of claim 1, wherein the psychiatric disorder is autism spectrum disorder, schizophrenia, or depression.

3. The method of claim 2, wherein the autism spectrum disorder is autism.

4. The method of claim 1, wherein the psychiatric disorder is associated with maternal inflammation.

5. The method of claim 1, wherein the inhibitor of Th17 cell activity is administered to the female in the first, the second or third trimester of the pregnancy.

6. The method of claim 1, wherein the female has elevated levels of IL-17a or other IL-17 family cytokine in her sera during the pregnancy with the fetus.

7. The method of claim 1, wherein the female was or is afflicted with a hyper-inflammatory condition during the pregnancy with the fetus.

8. The method of claim 7, wherein the hyper-inflammatory condition is associated with a viral or bacterial infection or exposure to an inflammatory or environmental toxin during the pregnancy with the fetus.

9. The method of any claim 7, wherein the inhibitor of Th17 cell activity is administered to the female during or after the hyper-inflammatory condition.

10. The method of claim 1, wherein the inhibitor of Th17 cell activity is an inhibitor of retinoic acid receptor-related orphan nuclear receptor gamma t (RORγt) activity and/or interleukin 17 (IL-17) activity or an enhancer of T regulatory (Treg) cell activity.

11. The method of claim 10, wherein the inhibitor of IL-17 activity is an antibody specific for a Th17 cell specific cytokine or a Th17 specific cell surface protein.

12. The method of claim 11, wherein the Th17 cell specific cytokine is IL-17f or IL-22, or wherein the Th17 specific cell surface protein is CCR6.

13. The method of claim 10, wherein the inhibitor of IL-17 activity is an antibody specific for IL-17a or IL-17 receptor.

14. The method of claim 13, wherein the antibody is a human monoclonal antibody or a humanized monoclonal antibody.

15. The method of claim 10, wherein the inhibitor of IL-17 activity is an antibody specific for the p19 subunit of IL-23, the p40 subunit of IL-23 and IL-12, or the IL-23 receptor.

16. The method of claim 1, wherein the inhibitor of Th17 cell activity is administered intravenously, subcutaneously, intraperitoneally, or orally.

17. The method of claim 1, wherein the inhibitor of Th17 cell activity does not transfer across the placenta or is modified to reduce or prevent transfer across the placenta.

18. The method of claim 1, wherein the female does not have a pre-existing condition associated with aberrant Th17 cell activity.

19. The method of claim 18, wherein the pre-existing condition associated with aberrant Th17 activity is multiple sclerosis, psoriasis, rheumatoid arthritis, or Crohn's Disease.

20. A method for decreasing the likelihood of a psychiatric disorder in a fetus, the method comprising administering an inhibitor of T helper 17 (Th17) cell activity to a female during a pregnancy wherein the female is carrying the fetus in utero.

21. A method for treating a pregnant female with a hyper-inflammatory condition, the method comprising administering an inhibitor of Th17 cell activity to the female while pregnant with a fetus to reduce inflammation in the pregnant female, thereby decreasing risk for developing a psychiatric disorder in the fetus.


SEQUENCE LISTING (22 sequences)

SEQ ID NO: 1 - DNA, 23 bp, Artificial Sequence
  primer
cccagcaggtaaatcagtggttc

SEQ ID NO: 2 - DNA, 22 bp, Artificial Sequence
  primer
gcggatagagcaaggtcattgg

SEQ ID NO: 3 - DNA, 25 bp, Artificial Sequence
  primer
gtaactgtgtttatgactccctggc

SEQ ID NO: 4 - DNA, 25 bp, Artificial Sequence
  primer
cactctttcttgacatctccccttc

SEQ ID NO: 5 - DNA, 24 bp, Artificial Sequence
  primer
ttccttccttcttcttgagcagtc

SEQ ID NO: 6 - DNA, 27 bp, Artificial Sequence
  primer
cagaagaaaagtatatgtggcttgttg

SEQ ID NO: 7 - DNA, 24 bp, Artificial Sequence
  primer
ggtcatttactggacaccctttcc

SEQ ID NO: 8 - DNA, 25 bp, Artificial Sequence
  primer
gctacacagcaaaaccttgtcttgg

SEQ ID NO: 9 - DNA, 20 bp, Artificial Sequence
  primer
acaagttggcccagcagaat

SEQ ID NO: 10 - DNA, 20 bp, Artificial Sequence
  primer
gggatatcaacaggctgcca

SEQ ID NO: 11 - DNA, 20 bp, Artificial Seqience
  primer
ccactctgtagcaccccaat

SEQ ID NO: 12 - DNA, 20 bp, Artificial Sequence
  primer
caggctccgtagttcctcag

SEQ ID NO: 13 - DNA, 20 bp, Artificial Sequence
  primer
ggtactgtccccaggggtat

SEQ ID NO: 14 - DNA, 20 bp, Artificial Sequence
  primer
gaggccggttttcatctcca

SEQ ID NO: 15 - DNA, 22 bp, Artificial Sequence
  primer
ctccagaaggccctcagactac

SEQ ID NO: 16 - DNA, 24 bp, Artificial Sequence
  primer
agctttccctccgcattgacacag

SEQ ID NO: 17 - DNA, 23 bp, Artificial Sequence
  primer
acacatgttctctgggaaatcgt

SEQ ID NO: 18 - DNA, 24 bp, Artificial Sequence
  primer
aagtgcatcatcgttgttcataca

SEQ ID NO: 19 - DNA, 20 bp, Artificial Sequence
  primer
ggctgtattcccctccatcg

SEQ ID NO: 20 - DNA, 22 bp, Artificial Sequence
  primer
ccagttggtaacaatgccatgt

SEQ ID NO: 21 - DNA, 21 bp, Artificial Sequence
  primer
aggtcggtgtgaacggatttg

SEQ ID NO: 22 - DNA, 23 bp, Artificial Sequence
  primer
tgtagaccatgtagttgaggtca